(19) United States
(12) Patent Application Publication
(10) Pub. No.: US 2006/0204993 A1
(43) Pub. Date: Sep. 14, 2006

(54) Methods and compositions for modulating CHK2 pathways and methods for screening for same

(76) Inventors: Chiang J. Li, West Roxbury, MA (US); Harry Rogoff, North Oxford, MA (US); Chang-Rung Chen, Newton, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/364,609

(22) Filed: Feb. 27, 2006

Related U.S. Application Data
(60) Provisional application No. 60/656,171, filed on Feb. 25, 2005.
(60) Provisional application No. 60/664,039, filed on Mar. 22, 2005.
(60) Provisional application No. 60/692,314, filed on Jun. 20, 2005.

(51) Int. Cl. C12Q 1/68; A61K 31/366

(52) U.S. Cl. 435/6; 514/454

(57) ABSTRACT

The present invention relates to a method for screening for an activator of Chk2. The screening method comprises assessing the Chk2-activating effect of a test compound in cells containing DNA encoding Chk2. The present invention also provides a method for the treatment or prevention of pre-cancers, cancers, or hyperproliferation disorders. The treating or preventing method comprises administering to a subject in need a therapeutically effective amount of a Chk2 activator.

### CROSS-REFERENCE TO RELATED APPLICATIONS

This application claims the benefit of U.S. Provisional Application No. 60/656,171, filed Feb. 25, 2005, No. 60/664,039 filed Mar. 22, 2005, and No. 60/692,314 filed Jun. 20, 2005, the contents of which are incorporated herein by reference in their entirety.

### BACKGROUND

Cancer chemotherapy and radiotherapy, which remain the mainstay treatment of most human cancers, primarily work by causing damage to DNA or microtubules, resulting in toxicity against cancer as well as normal cells with limited selectivity.

Complex mechanisms have evolved in mammalian cells to maintain genome integrity. DNA damage checkpoints are found to act as an anti-cancer barrier in early human carcinogenesis (Gorgoulis, et al. Nature 434:907-13 (2005)); Bartkova, et al. Nature 434:864-70 (2005)). Treatment of cells with DNA-damaging agents can lead to either activation of cell cycle checkpoints that stop DNA replication to allow for DNA repair, or apoptosis, with both mechanisms acting to ensure proper replication of DNA.

Defects in the genome integrity checkpoints allow cancer cells to evade cell death, DNA repair, and cell cycle arrest. Cancer cells are characterized by constitutive oncogenic stress, genomic instability, and accumulation of mutations and chromosomal aberrations (Lowe, et al., Nature 432, 307-15, 2004; Bartek, et al., Cancer Cell 3:421-9 (2003)). Nevertheless, the complex checkpoint network is partially preserved in cancer cells, presumably to prevent the accumulation of a lethal degree of genomic instability during uncontrolled cellular proliferation (Kastan and Bartek, Nature 432:316-23, 2004).

Chk2 (checkpoint kinase 2), a human checkpoint kinase, is thought to play a central role in the response to DNA damage (Kastan and Bartek, Nature 432:316-23 (2004)). Chk2 is able to receive signals from DNA damage sensors and relay those signals to the cell cycle machinery, resulting in a pause in cell cycle progression or, under certain circumstances, apoptosis (Kastan and Bartek, Nature 432:316-23 (2004); Bartek et al. Cancer Cell 3:421-9 (2003)). It appears that Chk2 forms a central component of DNA-damage response pathways to both DNA damage insults and telomere erosion.

Much of the data regarding the involvement of Chk2 in the DNA-damage-apoptosis pathway comes from studying the cellular response to DNA damage insults (Zhou and Elledge, Nature 408:433-9 (2000)). Agents that cause DNA double-strand breaks, including ionizing radiation (IR) and certain chemotherapeutic drugs, activate Chk2 by phosphorylation at threonine-68 mediated by the ataxia-telangiectasia mutated (Atm) kinase (Ahn et al., Cancer Res 60:5934-6 (2000); Melchionna et al., Nat Cell Biol, 2:762-765 (2000);

Schwarz et al., Mol Cancer Res 1:98-609 (2003)). Threonine-68 phosphorylation is followed by phosphorylation of the p53 tumor suppressor at the serine-20 residue, resulting in stabilization of p53 (Chehab et al., Genes Dev 14:278-288 (2000); Hirao et al., Science 287:1824-1827 (2000); Takai et al., Embo J, 21:5195-5205 (2002)), a p53-dependent increase in the cyclin-dependent kinase inhibitor p21 and, subsequently, a block in cell cycle progression. These findings suggest that Chk2 may play a pivotal role in the p53-regulated pathways that protect cells from replicating damaged DNA in the presence of DNA damage insults.

In addition to exogenous DNA-damaging insults, critical telomere erosions can generate an endogenous ‘DNA damage’ signal that leads to Chk2 activation. Most human somatic cells can undergo only a limited number of population doublings in vitro. This exhaustion of proliferative potential, termed senescence, can be triggered when telomeres cannot fulfill their normal protective functions. Senescence is a p53-dependent growth arrest state that is thought to represent an innate defense against tumor progression.

The therapeutic exploration of DNA-damage response pathways so far has been focused on developing checkpoint kinase inhibitors (Bartek et al. Cancer Cell 3:421-9 (2003)). It has been proposed that blocking activation of cell cycle checkpoint pathways in conjunction with administration of a DNA damaging agent would increase tumor cell sensitivity to the genotoxic agent, and limit normal cell death associated with chemotherapy.

Inhibiting crucial regulators of cell cycle checkpoint control, including Atm, Chk1, and Chk2 is currently being tested for sensitization of tumor cells to chemotherapy and/or radiation treatment (Zhou and Bartek, Nat Rev Cancer 4:216-25 (2004)). Inhibitors of Chk2 and other checkpoint kinases are now in preclinical and clinical development as sensitizers for chemotherapy (Kastan and Bartek, Nature 432:316-23 (2004); Bartek et al. Cancer Cell 3:421-9 (2003); Falck et al., Nat Genet 30:290-294 (2002); Jack et al., Proc Natl Acad Sci USA, 99:9825-9829 (2002)) and some success in tumor cell sensitization has been achieved by inactivation of the Chkl controlled cell cycle checkpoint pathways (Akinaga et al., Cancer Chemother Pharmacol 32:183-9 (1993); Koniaras et al., Oncogene 20:7453-63 (2001); Wang et al., Cancer Res 62:2483-7 (2002)).

The references cited herein are not admitted to be prior art to the claimed invention.

### SUMMARY OF THE INVENTION

The present invention provides a method for screening for an activator of Chk2. The method comprises the step of assessing the Chk2-activating effect of a test compound in cells containing DNA encoding Chk2. The step of assessing the Chk2-activating effect in cells can comprise exposing the cells to a test compound, measuring the activity of Chk2 in the cells in the presence and in the absence of the test compound, and comparing the activity of Chk2 in the presence and in the absence of the test compound. The Chk2-activating effect can be determined by an increase in the phosporylation of Chk2.

In an embodiment, the cells are cancer cells. The cancer cells can be cells in a cancer of a vertebrate, mammal, or human, or cells derived from a cancer of a vertebrate, mammal, or human. In a preferred embodiment, the cancer cells are cultured cancer cells. The cultured cancer cells can be selected from the group consisting of A549, NCI-H460, NCI-H1299, DLDI, HT-29, and HCT-1 16 cells.

In an embodiment, the test compound is a small molecule, preferably an analog, derivative, or metabolite of β-lapachone.

The method for screening for an activator of Chk2 can further comprise the step of assessing the Chk2-activating effect of the test compound in normal cells containing DNA encoding Chk2. The step of assessing the Chk2-activating effect in normal cells can comprise exposing the normal cells to a test compound, measuring the activity of Chk2 in the normal cells in the presence and in the absence of the test compound, and comparing the activity of Chk2 in the presence and in the absence of the test compound. The normal cells can be normal cells in a vertebrate, mammal, or human or normal cells derived from a vertebrate, mammal, or human. In an embodiment, the normal cells are cultured normal cells. The cultured normal cells can be selected from the group consisting of IMR-90, MCF-10A, NCM460, and PBMC cells.

The method for screening for an activator of Chk2 can further comprise the step of selecting the test compound that has a higher Chk2-activating effect in the cancer cells than in the normal cells.

Alternatively, the method for screening for a Chk2 activator comprises the step of assessing the Chk2-activating of a test compound in the lysate of cells containing DNA encoding Chk2. The cells can be cancer cells. The method can further comprises assessing the Chk2-activating effect of the test compound in the lysate of normal cells containing DNA encoding Chk2, and comparing the Chk2-activating effects of the test compound in the cancer cell lysate and the normal cell lysate.

In another embodiment, the method for screening for a Chk2 activator comprises contacting Chk2 with a test compound, measuring the activity of Chk2 in the presence and in the absence of the test compound, and comparing the activity of Chk2 in the presence and in the absence of the test compound. The method can further comprises selecting the test compound that increases the Chk2 activity. The method can further comprises assessing the Chk2-activating effect of the selected compound in cancer cells containing DNA encoding Chk2, or the lysate of the cells, assessing the Chk2-activating effect of the selected compound in the lysate of normal cells containing DNA encoding Chk2, or the lysate, and comparing the Chk2-activating effects of the selected compound in the cancer cells or the lysate and the normal cells or the lysate.

The present invention also provides a method of treating or preventing cancer in a subject. The method comprises increasing Chk2 activity in cancer cells of the subject in the absence of de novo DNA damage. In an embodiment, the method comprises selectively increasing Chk2 activity in cancer cells of the subject.

In a preferred embodiment, the method comprises administering to the subject a therapeutically effective amount of a Chk2 activator that is not β-lapachone, preferably a selective activator of Chk2. The compound can be an analog, derivative, or metabolite of β-lapachone. The subject can a vertebrate, mammal, or human.

In a further preferred embodiment, the method of treating or preventing cancer in a subject comprising administering to the subject a therapeutically effective amount of a Chk2 activator that is identified by the method for screening for an activator of Chk2.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1A is a photograph of immunoblots showing total Chk2 and the phospho-threonine 68 form of Chk2, in total cellular lysates following tetracycline treatment. FIG. 1B is a graph of MTT assays showing that induction of CHK2 expression was found to suppress cell proliferation. FIG. 1C is a photograph of colony assays showing that induction of CHK2 expression was found to suppress cell proliferation.

FIG. 2A is a graph showing the DNA contents of cells as measured by flow cytometry (upper panel) and the percentages of Annexin-V-positive cells and Pancaspase-positive cells determined by flow cytometric analysis (bottom panels). FIG. 2B is a photograph of fluorescent microscopy showing DLD1-tet-Chk2# 17 cells cultured in the presence or absence of tetracycline following staining with DePsipher Dye (Trevigen) and monitoring the intensity of the monomer (530 nM) and aggregates (590 nM) of DePsipher. FIG. 2C is a photograph of immunoblots showing DLD1-tet-Chk2 #17 cell lysates following tetracycline treatment. Lysates were prepared and subjected to immunoblotting with the antibodies indicated on the left. FIG. 2D is a series of graphs of flow cytometric analysis showing the DNA content, caspase activity and percentages of DLD1-tet-Chk2#17 cells undergoing apoptosis following treated with Z-VAD- or mock-treated (None) and cultured in the presence (+tet) or absence (−tet) of tetracycline for six days.

FIG. 3A is a photograph of senescence-associated beta-galactosidase staining of DLD1-tet-Chk2#17 cells grown in the presence (+tet) or absence (−tet) of tetracycline. FIGS. 3B and 3C and photographs of immunofluorescence staining of DLD1-tet-Chk2#17 cells cultured in the presence (+tet) or absence (−tet) of tetracycline with antibodies indicated. The merged images of two different stainings are shown (Merge).

FIG. 4. Chk2 expression and activation in tumor cell lines. FIG. 4A, colon cell lines were infected with AdChk2 at a multiplicity of infection (MOI) of 25. Cells were harvested and extracts made at 24 hours post infection (hpi). Western blot analysis was performed to determine levels of Chk2 and the phospho-threonine 68 form of Chk2. Actin blots are shown as protein loading controls. FIG. 4B, lung cell lines were infected with AdChk2 at a multiplicity of infection (MOI) of 25. Cells were harvested and extracts made at 24 hours post infection (hpi). Western blot analysis was performed to determine levels of Chk2 and the phospho-threonine 68 form of Chk2. Actin blots are shown as protein loading controls.

FIG. 5. Selective induction of apoptosis in cancer cells by Chk2 activation. FIG. 5A, colon cell lines were infected with AdCon or AdChk2 at the indicated MOI. Cells were harvested and DNA stained with propidium iodide (PI) at 48 hpi. Number in each panel represents percentage of cells with sub-G1 DNA content. FIG. 5B, lung cell lines were infected with AdCon or AdChk2 at the indicated MOI. Cells were harvested and DNA stained with propidium iodide (PI) at 48 hpi. Number in each panel represents percentage of cells with sub-G1 DNA content.

FIG. 6. Role of survivin in Chk2-induced apoptosis. FIG. 6A, western blot analysis of survivin protein levels in colon and lung cells infected with AdCon or AdChk2 at an MOI of 25. Cells were harvested and extracts made at 16 hpi. Actin blots are shown as protein loading controls. FIG. 6B, western blot analysis of Chk2 and survivin protein levels in SW-480 colon cancer cells transfected with either siCon or siChk2 and harvested at the indicated times following transfection. Actin blots are shown as protein loading controls. FIG. 6C, analysis of cell death in DLD1 and A549 cells transfected with either pcDNA or pcDNA-survivin prior to infection with AdCon or AdChk2. Cells were harvested and MTT assay performed at 48 hpi. Error bars represent standard deviation of experiment performed in triplicate.

FIG. 7. Chk2 expression inhibits tumor growth in vivo. FIG. 7A, western blot analysis was performed to determine levels of Chk2 and the phospho-threonine 68 form of Chk2 in Dld1-Chk2 inducible cell line following addition of 1000 ng/ml tetracycline at time 0. Cells were harvested and extracts were made at indicated time points following addition of tetracycline to the growth medium. FIG. 7B, DLD1-Control and DLD1-Chk2 cells were cultured in the presence or absence of tetracycline for 24 h. Then 4×10⁶ cells were harvested and injected subcutaneously into the flank region of female nude mice. The mice were fed water with (+) or without (−) 1 mg of tetracycline per ml. Tumor volumes (mm³) were monitored for 34 days. The change in tumor volume over a 34-day period is shown in the graph. Error bars represent standard deviations of tumor volumes. FIG. 7C, representative tumors are shown from the four groups of mice from panel B. FIG. 7D, DLD1-Chk2 cells were injected subcutaneously into the flank region of female nude mice. When tumors reached volumes indicated by arrows (100 and 300 mm³), tetracycline was added to drinking water and tumor volumes (mm³) were monitored for 32 days. The change in tumor volume over a 32-day period is shown in the graph. Error bars represent standard deviations of tumor volumes.

FIG. 8. Chk2 expression results in decreased survivin in vivo. Immunohistochemical staining of phospho-threonine 68 Chk2 and survivin protein levels in xenograft tumor sections derived from DLD1 or DLD1-Chk2 (+tetracycline) cell lines. FIG. 9. Correlation between phospho-Chk2 and survivin in human tumor samples. FIG. 9A, immunohistochemical staining of phospho-threonine 68 Chk2 and survivin protein levels in early stage breast cancer sections. FIG. 9B, table showing relative staining levels of phospho-threonine 68 Chk2 and survivin in early stage breast cancer samples.

### DETAILED DESCRIPTION

The present invention relates to a method to screen for a Chk2 activator that activates Chk2 in the absence of de novo DNA damage, and the use of the Chk2 activator in the prevention and treatment of cancer.

### 1. THE ACTIVATION OF CHK2 IN THE ABSENCE OF DE NOVO DNA DAMAGE

Normally, the Chk2 pathway is activated by DNA damage caused by DNA-damaging agents. Responsive to DNA damage, Chk2 is activated through phosphorylation at the threonine 68 residue mediated primarily by the ataxia telangiectasia mutated (Atm) kinase (Matsuoka et al., Science 282:1893-7 (1998); Matsuoka et al. Proc Natl Acad Sci USA 97:10389-94 (2000); Melchionna et al., Nat Cell Biol 2:762-5 (2000); Chaturvedi et al., Oncogene 18:4047-54 (1999)). The initial Atm-dependent phosphorylation event is followed by oligomerization of Chk2 and autophosphorylation at multiple residues, leading to increased Chk2 kinase activity (Xu et al., Mol Cell Biol 22:4419-32 (2002); Wu and Chen, J Biol Chem 278:36163-8 (2003); Ahn et al., J Biol Chem 277:19389-95 (2002)). Overexpression of Chk2 also leads to enzymatic activation through autophosphorylation (Ng et al., J Biol Chem 279:8808-19 (2004)).

As described above, inhibitors of Chk2 and other checkpoint kinases are in preclinical and clinical development as sensitizers for chemotherapy. In contrast, the present invention describes that the Chk2 activation, in the absence of de novo DNA damage, leads to potent inhibition of survival and proliferation of cancer cells. The present invention provides a Chk2 activator that activates Chk2 in cells without causing de novo DNA damage. As described herein, the Chk2 activation in the absence of de novo DNA damage selectively kills cancer cells. Without de novo DNA damage, Chk2 activation induces cell death in a broad panel of malignant cells, while having little effects on normal cells.

As used herein, “de novo DNA damage” refers to the primary DNA damage caused by traditional chemotherapy and radiotherapy. Chemotherapy and radiotherapy causes DNA damage in cells, both cancer cells and normal cells, which consequently leads to cell death of the cells. In contrast, Chk2 activation does not cause the primary DNA damage. The Chk2 activation in cancer cells leads to cell death of the cancer cells, which consequently causes DNA damage. Thus, the DNA damage caused by Chk2 activation is a secondary event to the cell death of the cancer cells. Moreover, Chk2 activation does not cause cell death of normal cells.

### 2. THE METHOD FOR THE SCREENING OF CHK2 ACTIVATOR

The present invention provides a method for screening for a Chk2 activator comprising the step of assessing the Chk2-activating effect of a test compound. As used herein, the “Chk2-activating effect of a test compound” refers to the capability of a test compound to increase Chk2 activity. The terms “increase”, “enhance”, “induce” or “promote” are used interchangeably herein. Further, the terms “decrease”, “reduce”, “inhibit” or “prevent” are used interchangeably herein.

The Chk2 activity may be determined by the measurement of Chk2 phosphorylation at the threonine 68 residue. The measurement of Chk2 activity is known in the art (see, e.g., Melchionna, et al., Nat Cell Biol 2:762-5 (2000); Ahn, et al., Cancer Res 60:5934-6 (2000)). The test compound that increases the Chk2 activity is a Chk2 activator.

The Chk2-activating effect of a test compound may be measured by the ratio of the Chk2 activity in the presence and in the absence of the test compound. In an embodiment, the Chk2 activity in the presence of the test compound is about 1.5 fold, about 2 fold, about 4 fold, about 10 fold, about 20 fold, about 40 fold, about 100 fold, about 200 fold, about 500 fold, about 1,000 fold, or more than 1,000 fold of the Chk2 activity in the absence of the test compound.

A Chk2 activator may increase the Chk2 activity via various mechanisms. A Chk2 activator may increase the transcription, post-transcription, translation, or translocation of Chk2, or the combination of the above. A Chk2 activator may interact with Chk2 directly, or with the modulator(s) of Chk2, or with both. In an embodiment, methods of screening Chk2 activator comprising screening compounds for their ability to increase the activity or expression of Chk2.

The Chk2-activating effect of a test compound can be assessed using various systems, such as animal model, cultured cells, cell lysate, or isolated Chk2, or the combination of the above.

### 2.1. The Assessing Method Using Cells

In an embodiment, the method for screening for a Chk2 activator comprises the step of assessing the Chk2-activating effect of a test compound in cells. The cells used for the assessment should contain DNA encoding Chk2. The cells can be in vivo, in vitro or ex vivo. The step of assessing the Chk2-activating effect may comprise exposing the cells to a test compound, measuring the kinase activity of Chk2 in the cells in the presence and in the absence of the test compound, and comparing the Chk2 activity in the presence and in the absence of the test compound. The test compound that increases the Chk2 activity may then be selected as a Chk2 activator.

In another embodiment, the method of screening Chk2 activator comprising measuring the ability of the test compound to increase the activity or expression of Chk2. In one embodiment, this method includes exposing cells expressing Chk2 with a test compound, and then measuring the expression of Chk2. The expression of Chk2 in the presence of the test compound is then compared to the expression of Chk2 in the absence of the test compound. If the expression of Chk2 in the presence of the test compound is more than the expression of Chk2 in the absence of the test compound, then the test compound is a Chk2 activator that induces or promotes Chk2 expression. The expression can be measured by any means known in the art, for 35 example, Western blotting. The expression of Chk2 in cells not exposed with the test compound can be about 0%, about 1%, about 10%, about 20%, about 50%, or about 75% of the expression in cells exposed with the test compound.

The cells used for the assessment can be cells directly from a eukaryotic organism, preferably vertebrate, more preferably mammal, and further preferably human. Alternatively, the cells used for the assessment can be cultured cells.

The cells used for the assessment are preferably cancer cells. As used herein, “cancer cells” refer to cells that are derived from primary, metastatic, or blood-borne cancers directly from vertebrate, preferably mammal, more preferably human. The cancer cells will in most cases, but not exclusively, be characterized as displaying the so-called “transformed phenotype”, harboring a genetic defect that confers upon said cells unlimited replicative potential, and additionally exhibiting the ability to grow in an anchorage-independent manner in semi-solid tissue culture medium (soft agar, e.g.) and characterized by the ability to form subcutaneous tumors when injected or implanted into immunologically compromised or sub-lethally irradiated rodents or other animal models. The cancer cells can be cells in a cancer from a vertebrate, mammal, or human. The cancer cells can also be cells in a cancer from a chimera animal, or cells derived from a cancer in a chimera animal. One example of the chimera animal is mice with human xenograft tumors (see, e.g., Calabrese et al, J Natl. Cancer Inst. 96:56-67 (2004).

Alternatively, the cancer cells may be cultured cancer cells, propagated indefinitely as adherent monolayers in sterile polystyrene plates. The cultured cancer cells can be cell lines of breast cancer, colon cancer, lung cancer, melanoma, pancreatic cancer, ovarian cancer, and prostate cancer cell lines. Examples of the cultured cancer cells include MCF-7 (human breast cancer cells), HT-29, HCT-116, DLD1, and SW480 (human colonic cells); Paca-2 (human pancreatic cancer cells); A549, NCI-H460, and NCI-H1299 (lung cancer cell lines).

Preferably, the Chk2 activator is a selective activator of Chk2, which increases Chk2 activity in cancer cells more than in normal cell. In order to screen for the selective activator of Chk2, the method of for screening for a Chk2 activator may further comprise the step of assessing the Chk2-activating effect of the test compound in normal cells, as well as in cancer cells. The step of assessing the Chk2-activating effect in normal cells may comprise exposing the normal cells to a test compound, measuring the activity of Chk2 in the normal cells in the presence and in the absence of the test compound, and comparing the activity of Chk2 in the presence and in the absence of the test compound. The test compound that increases the Chk2 activity in the cancer cells more than in the normal cells may then be selected as a selective activator of Chk2.

The selectivity of a Chk2 activator may be measured by the ratio of Chk2-activating effects of a test compound in cancer cells and in normal cells. In an embodiment, the Chk2-activating effect of a test compound in cancer cells is about 1.5 fold, about 2 fold, about 4 fold, about 10 fold, about 20 fold, about 40 fold, about 100 fold, about 200 fold, about 500 fold, about 1,000 fold, or more than 1,000 fold of the Chk2-activating effect of a test compound in cancer cells.

As used herein, “normal cells” refer to cells that have a limited replicative potential relative to cancer cells and that will cease to divide in culture after a finite number of cell divisions. These normal cells will encompass cells that do not exhibit the so-called “transformed phenotype” of cancer cells, will not grow in an anchorage-independent manner in semi-solid tissue culture medium (soft agar, e.g.) and will not form subcutaneous tumors when injected or implanted into immunologically compromised or sub-lethally irradiated rodents or other animal models. The normal cells may be cells directly isolated from tissues of vertebrates, preferably mammal, more preferably human, such as human dermal fibroblasts from skin biopsies, proliferating peripheral blood mononuclear cells (PBMC) isolated from whole blood, or human epithelial cells isolated from normal breast tissue following reduction mammoplasty. The normal cells can be normal cells in a vertebrate, mammal, or human. Alternatively, the normal cells may be cultured cell lines that have been propagated in vitro and have acquired an increased replicative potential (become “immortalized”) without adopting the transformed phenotype, such as MCF-10A (nontransformed breast epithelial cells), NCM460 (normal colonic epithelial cells), and IMR-90 (nontransformed lung fibroblasts).

In the method screening for selective activator of Chk2, the cancer cells and normal cells preferably share some major characteristics. For examples, see Li, et al., PNAS 100:2674-2678 (2003).

2.2. The Assessing Method Using Cell Lysate

In another embodiment, the method for screening for a Chk2 activator comprises the step of assessing the Chk2-activating effect of a test compound in lysate of cells. The cells used for the assessment should contain DNA encoding Chk2. The step of assessing the Chk2-activating effect may comprise exposing the cell lysate to a test compound, measuring the Chk2 activity in the cell lysate in the presence and in the absence of the test compound, and comparing the Chk2 activity in the presence and in the absence of the test compound. The compound that increases the Chk2 activity may then be selected as a Chk2 activator.

Cell lysates may be generated by various methods known by the skill of the art. For examples see Current protocols in protein science, John E. Coligan et al., Publisher: New York: Wiley 1995-2002 Edition: (v. 1)

The assessing method preferably uses the lysate of cancer cells. The cancer cells are those described in section 1.1.

Similarly, the Chk2-activating effects of a test compound in cancer cell lysate and in normal cell lysate may be compared. The test compound that increases the Chk2 activity in the cancer cell lysate more than in the normal cell lysate may then be selected as a selective activator of Chk2. The selectivity of a Chk2 activator may be determined as in section 1.1.

2.3. The Assessing Method Using Chk2

In another embodiment, the method for screening for a Chk2 activator comprises contacting Chk2 with a test compound, measuring the activity of Chk2 in the presence and in the absence of the test compound, and comparing the activity of Chk2 in the presence and in the absence of the test compound. The test compound that increases the Chk2 activity may then be selected as a Chk2 activator. In one embodiment, 5 Chk2 is a protein isolated from Homo sapiens, with the amino acid sequence as disclosed in Genbank Accession No. ACC48504. Chk2 also refers to similar proteins isolated in other organisms, for example, Mus musculus (GenBank Accession No. AAC83694) or Rattus norvegicus (GenBank Accession No. AAD55890).

Once a test compound is selected as a Chk2 activator, the activator may be further tested for its selectivity in Chk2 activation. The further test may follow the procedures described above. So the method may further comprise assessing the Chk2-activating effect of the activator in cancer cells containing DNA encoding Chk2, or the lysate of the cancer cells, assessing the Chk2-activating effect of the activator in normal cells containing DNA encoding Chk2, or the lysate of the normal cells, and comparing the Chk2-activating effects of the activator in the cancer cells or lysate and the normal cells or lysate. The test compound that increases the Chk2 activity in the cancer cells or lysate more than in the normal cells or lysate may then be selected as a selective activator of Chk2. The selectivity of a Chk2 activator may be determined as in section 1.1.

2.4. The method for screening for cancer drug candidate

The present invention also provides a method (also referred to herein as a “screening assay”) for identifying candidate or test compounds or agents that have an enhancing effect on Chk2 activation or expression and therefore promote cell death of cancer cells. The present invention also includes compounds identified in the screening assays described herein.

In yet another embodiment, the present invention is directed to a method for identifying a potential therapeutic agent for use in the treatment of pre-cancer or cancer, the method comprising providing cells, tissues, or animals; exposing the cells, tissues, or animals to a composition comprising a test compound, wherein the test compound enhances Chk2 activity or expression; and monitoring the progression of the pre-cancer or cancer; wherein, if the progression of the pre-cancer or cancer is reduced, the candidate compound is identified as a potential therapeutic agent.

In one embodiment, an assay is a cell-based assay in which a cancer cell is exposed to a test compound and the ability of the test compound to enhance activation or expression of Chk2 directly or indirectly and reduce the progression of pre-cancer or cancer is determined. The cell, for example, can be of mammalian or human origin, and could be a pre-cancer or cancer cell. Determining the ability of the test compound to reduce the progression of pre-cancer or cancer can be accomplished, for example, by monitoring the progression of the pre-cancer or cancer.

The present invention also provides a method for monitoring the effectiveness of treatment of a subject with a test compound or an agent which enhances the activation or expression of Chk2 directly or indirectly, comprising the steps of (i) obtaining a pre-administration sample from a subject prior to administration of the agent; (ii) detecting the level of expression or activity of Chk2 in pre-cancer or cancer cells in the preadministration sample; (iii) obtaining one or more post-administration samples from the subject; (iv) detecting the level of expression or activity of Chk2 pre-cancer or cancer cells in the post-administration samples; (v) comparing the level of expression or activity Chk2 of the pre-cancer or cancer cells in the pre-administration sample with the pre-cancer or cancer cells in the post administration sample or samples; and (vi) altering the administration of the agent to the subject accordingly.

Suitable in vitro or in vivo assays can be performed to determine the effect of a composition which enhances Chk2 activity or expression and whether its administration inhibits growth of pre-cancer or cancer cells. In various specific embodiments, in vitro assays may be performed with representative pre-cancer or cancer cells, to determine if a given therapeutic exerts the desired effect upon the cell type(s). Compounds for use in therapy may be tested in suitable animal model systems including, but not limited to rats, mice, cows, monkeys, rabbits, and the like, prior to testing in human subjects. Similarly, for in vivo testing, any of the animal model system known in the art may be used prior to administration to human subjects.

### 3. THE TEST COMPOUNDS

The test compound can be protein, peptide, peptidomimetic, nucleic acid, small molecule, or other drug candidates. The test compound should not cause de novo DNA damage in cells. If it is unknown whether a test compound is capable of causing de novo DNA damage in cells, the compound can be tested for such a capability. Such a test can be done before or after the compound is tested for its capability of activating Chk2. In an embodiment, Chk2 activation, cell death, and DNA damage are measured in the cells contacted with the test compound. If Chk2 activation occurs before cell death and DNA damage, the test compound does not cause de novo DNA damage.

In a preferred embodiment, the test compound is a small molecule. A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention. In a preferred embodiment, the small molecule used in the present invention is β-lapachone or an analog, derivative, or metabolite thereof.

The increase in the expression of Chk2 can also be achieved with other approaches. Specifically, the methods include anti-sense and RNA interference (RNAi), along with methods of heterologously expressing Chk2 in a cell. Specific siRNAs and antisense nucleotides for the modulation of expression of Chk2 are also included in the invention.

3. 1. The Libraries of the Test Compounds

The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, Anticancer Drug Design 12:145 (1997).

Examples of methods for the synthesis of molecular libraries can be found in the art, for example in:

DeWitt, et al., PNAS 90:6909 (1993); Erb, et al., PNAS 91:11422 (1994); Zuckermann, et al., J. Med. Chem. 37:2678 (1994); Cho, et al., Science 261:1303 (1993); Carrell, et al., Angew. Chem. Int. Ed. Engl. 33:2059 (1994); Carell, et al., Angew. Chem. Int. Ed. Engl. 33:2061 (1994); and Gallop, et al., J. Med. Chem. 37:1233 (1994).

Libraries of compounds may be presented in solution (e.g., Houghten, Biotechniques 13:412-421 (1992)), or on beads (Lam, Nature 354:82-84 (1991)), on chips (Fodor, Nature 364:555-556 (1993)), bacteria (Ladner, U.S. Pat. No. 5,223,409), spores (Ladner, U.S. Pat. No. 5,233,409), plasmids (Cull, et al., PNAS, 89:1865-1869 (1992)) or on phage (Scott and Smith, Science 249:386-390 (1990); Devlin, Science 249:404-406 (1990); Cwirla, et al., PNAS 87:6378-6382 (1990); Felici, J Mol. Biol. 222:301-310 (1991); Ladner, U.S. Pat. No. 5,233,409.).

3.2 The Analog, Derivative, or Metabolite of β-lapachone

In one embodiment, the candidate compound is a β-lapachone analog, derivative or metabolite. As further used herein, the phrase “β-lapachone” refers to 3,4-dihydro-2,2-dimethyl-2H-naphtho [1,2-b]pyran-5,6-dione, and has the chemical structure:

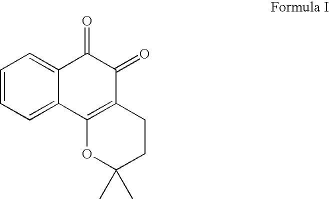

β-lapachone, or analogs, derivatives or metabolites thereof, in accordance with the present invention, can be synthesized as described in U.S. Pat. No. 6,458,974, which is incorporated by reference herein in its entirety. Preferred derivatives and analogs are discussed below.

In another embodiment, analogs of β-lapachone include reduced β-lapachone (Formula Ia, in which R′ and R″ are each hydrogen), as well as derivatives of reduced beta-lapachone (Formula Ia, in which R′ and R″ are each independently hydrogen, lower alkyl, or acyl).

In yet another embodiment, β-lapachone derivatives or analogs, such as lapachol, and pharmaceutical compositions and formulations thereof are part of the present invention. Such β-lapachone 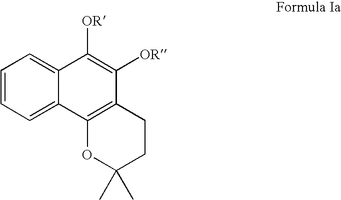
analogs include, without limitation, those recited in PCT International Application PCT/US93/07878 (WO 94/04145), which is incorporated by reference herein in its entirety, and which discloses compounds of the formula: 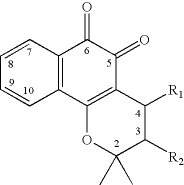
where R₁ and R₂ are each independently hydrogen, substituted and unsubstituted aryl, substituted and unsubstituted alkenyl, substituted and unsubstituted alkyl and substituted or unsubstituted alkoxy. The alkyl groups preferably have from 1 to about 15 carbon atoms, more preferably from I to about 10 carbon atoms, still more preferably from 1 to about 6 carbon atoms. The term alkyl unless otherwise modified refers to both cyclic and noncyclic groups, although of course cyclic groups will comprise at least three carbon ring members. Straight or branched chain noncyclic alkyl groups are generally more preferred than cyclic groups. Straight chain alkyl groups are generally more preferred than branched. The alkenyl groups preferably have from 2 to about 15 carbon atoms, more preferably from 2 to about 10 carbon atoms, still more preferably from 2 to 6 carbon atoms. Especially preferred alkenyl groups have 3 carbon atoms (i.e., 1-propenyl or 2-propenyl), with the allyl moiety being particularly preferred. Phenyl and napthyl are generally preferred aryl groups. Alkoxy groups include those alkoxy groups having one or more oxygen linkage and preferably have from 1 to 15 carbon atoms, more preferably from 1 to about 6 carbon atoms. The substituted R₁ and R₂ groups may be substituted at one or more available positions by one or more suitable groups such as, for example, alkyl groups such as alkyl groups having from 1 to 10 carbon atoms or from I to 6 carbon atoms, alkenyl groups such as alkenyl groups having from 2 to 10 carbon atoms or 2 to 6 carbon atoms, aryl groups having from six to ten carbon atoms, halogen such as fluoro, chloro and bromo, and N, O and S, including heteroalkyl, e.g., heteroalkyl having one or more hetero atom linkages (and thus including alkoxy, aminoalkyl and thioalkyl) and from I to 10 carbon atoms or from 1 to 6 carbon atoms.

Other β-lapachone analogs contemplated in accordance with the present invention include those described in U.S. Pat. No. 6,245,807, which is incorporated by reference herein in its entirety, and which discloses β-lapachone analogs and derivatives having the structure: 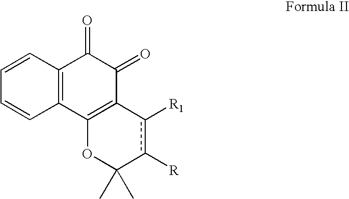
where R and R₁ are each independently selected from hydrogen, hydroxy, sulfhydryl, halogen, substituted alkyl, unsubstituted alkyl, substituted alkenyl, unsubstituted alkenyl, substituted aryl, unsubstituted aryl, substituted alkoxy, unsubstituted alkoxy, and salts thereof, where the dotted double bond between the ring carbons represents an optional ring double bond.

Additional β-lapachone analogs and derivatives are recited in PCT International Application PCT/US00/10169 (WO00/61142), which is incorporated by reference herein in its entirety, and which discloses compounds of the structure: 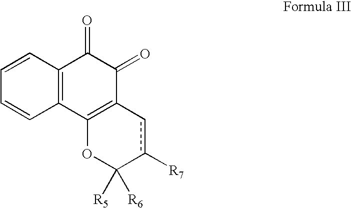
where R₅ and R₆ may be independently selected from hydroxy, C₁-C₆ alkyl, C₁-C₆ alkenyl, C₁-C₆ alkoxy, C₁-C₆ alkoxycarbonyl, —(CH₂)ₙ-phenyl; and R₇ is hydrogen, hydroxyl, C₁-C₆ alkyl, C₁-C₆ alkenyl, C₁-C₆ alkoxy, C₁-C₆ alkoxycarbonyl, —(CH₂)ₙ-amino, —(CH₂)ₙ-aryl, —(CH₂)ₙ-heteroaryl, —(CH₂)ₙ-heterocycle, or —(CH₂)ₙ-phenyl, wherein n is an integer from 0 to 10.

Other β-lapachone analogs and derivatives are disclosed in U.S. Pat. No. 5,763,625, U.S. Pat. No. 5,824,700, and U.S. Pat. No. 5,969,163, as well is in scientific journal articles, such as Sabba et al., J Med Chem 27:990-994 (1984), which discloses β-lapachone with substitutions at one or more of the following positions: 2-, 8- and/or 9- positions. See also Portela et al., Biochem Pharm 51:275-283 (1996) (substituents at the 2- and 9- positions); Maruyama et al., Chem Lett 847-850 (1977); Sun et al., Tetrahedron Lett 39:8221-8224 (1998); Goncalves et al., Molecular and Biochemical Parasitology 1:1 67-176 (1998) (substituents at the 2- and 3- positions); Gupta et al., Indian Journal of Chemistry 16B: 35-37 (1978); Gupta et al., Curr Sci 46:337 (1977) (substituents at the 3- and 4- positions); DiChenna et al., J Med Chem 44:2486-2489 (2001) (monoarylamino derivatives). Each of the above-mentioned references are incorporated by reference herein in their entirety.

More preferably, β-lapachone analogs and derivatives contemplated by the present application are intended to encompass compounds having the general formula II and III: 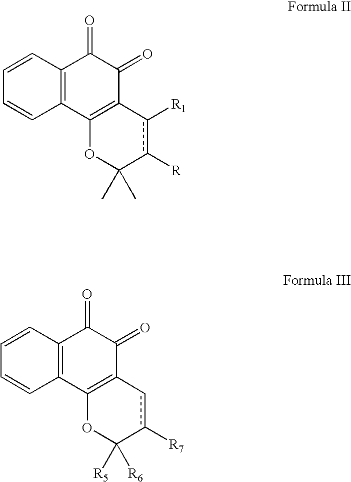
where the dotted double bond between the ring carbons represents an optional ring double bond and where R and R₁ are each independently selected from hydrogen, hydroxy, sulfhydryl, halogen, substituted alkyl, unsubstituted alkyl, substituted alkenyl, unsubstituted alkenyl, substituted aryl, unsubstituted aryl, substituted alkoxy, unsubstituted alkoxy, and salts thereof. The alkyl groups preferably have from 1 to about 15 carbon atoms, more preferably from 1 to about 10 carbon atoms, still more preferably from 1 to about 6 carbon atoms. The term alkyl refers to both cyclic and noncyclic groups. Straight or branched chain noncyclic alkyl groups are generally more preferred than cyclic groups. Straight chain alkyl groups are generally more preferred than branched. The alkenyl groups preferably have from 2 to about 15 carbon atoms, more preferably from 2 to about 10 carbon atoms, still more preferably from 2 to 6 carbon atoms. Especially preferred alkenyl groups have 3 carbon atoms (i.e., 1-propenyl or 2-propenyl), with the allyl moiety being particularly preferred. Phenyl and napthyl are generally preferred aryl groups. Alkoxy groups include those alkoxy groups having one or more oxygen linkage and preferably have from 1 to 15 carbon atoms, more preferably from 1 to about 6 carbon atoms. The substituted R and R₁ groups may be substituted at one or more available positions by one or more suitable groups such as, for example, alkyl groups having from 1 to 10 carbon atoms or from 1 to 6 carbon atoms, alkenyl groups having from 2 to 10 carbon atoms or 2 to 6 carbon atoms, aryl groups having from six to ten carbon atoms, halogen such as fluoro, chloro and bromo, and N, O and S, including heteroalkyl, e.g., heteroalkyl having one or more hetero atom linkages (and thus including alkoxy, aminoalkyl and thioalkyl) and from 1 to 10 carbon atoms or from 1 to 6 carbon atoms; and where R₅ and R6 may be independently selected from hydroxy, C₁-C₆ alkyl, C₁-C₆ alkenyl, C₁-C₆ alkoxy, C₁-C₆ alkoxycarbonyl, —(CH₂)ₙ-aryl, —(CH₂)ₙ-heteroaryl, —(CH₂)ₙ-heterocycle, or —(CH₂)ₙ-phenyl; and R₇ is hydrogen, hydroxyl, C₁-C₆ alkyl, C₁-C₆ alkenyl, C₁-C₆ alkoxy, C₁-C₆ alkoxycarbonyl, —(CH₂)ₙ-amino, —(CH₂)ₙ-aryl, —(CH₂)ₙ-heteroaryl, —(CH₂)ₙ -heterocycle, or —(CH₂)ₙ-phenyl, wherein n is an integer from 0 to 10.

Preferred β-lapachone analogs and derivatives also contemplated by the present invention include compounds of the following general formula IV: 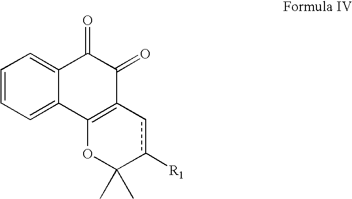
where R₁is (CH₂)ₙ-R₂, where n is an integer from 0-10 and R₂ is hydrogen, an alkyl, an aryl, a heteroaromatic, a heterocyclic, an aliphatic, an alkoxy, an allyloxy, a hydroxyl, an amine, a thiol, an amide, or a halogen.

Analogs and derivatives also contemplated by the present invention include 4-acetoxy-β-lapachone, 4-acetoxy-3-bromo-β-lapachone, 4-keto-β-lapachone, 7-hydroxy-β-lapachone, 7-methoxy-β-lapachone, 8-hydroxy-β-lapachone, 8-methoxy-β-lapachone, 8-chloro-β-lapachone, 9-chloro-β-lapachone, 8-methyl-β-lapachone and 8,9-dimethoxy-β-lapachone.

Other β-lapachone analogs and derivatives also contemplated by the present invention include compounds of the following general formula V: 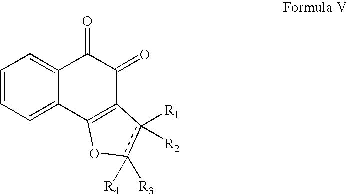
where R₁-R₄ are each, independently, selected from the group consisting of H, C₁-C₆ alkyl, C₁-C₆ alkenyl, C₁-C₆ alkoxy, C₁-C₆ alkoxycarbonyl, —(CH₂)ₙ-aryl, —(CH₂)ₙ-heteroaryl, —(CH₂)ₙ-heterocycle, or —(CH₂)ₙ-phenyl; or R₁ and R₂ combined are a single substituent selected from the above group, and R₃ and R₄ combined are a single substituent selected from the above groups, in which case—is a double bond.

Preferred β-lapachone analogs and derivatives also contemplated by this invention include dunnione and 2-ethyl-6-hydroxynaphtho[2,3-b]-furan-4,5-dione.

Preferred β-lapachone analogs and derivatives also contemplated by the present invention include compounds of the following general formula VI: 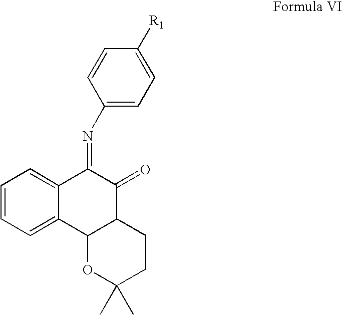
where R₁ is selected from H, CH₃, OCH₃ and NO₂.

Additional preferred P-lapachone analogs useful in the methods and kits of the present invention are recited in PCT International Application PCT/US03/37219 (WO2004/045557), which is incorporated by reference herein in its entirety, and which discloses compounds represented by Formula VII: 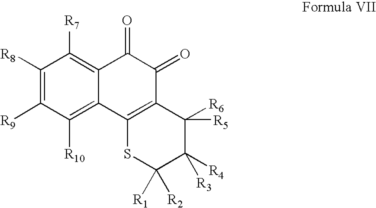
or pharmaceutically acceptable salts thereof, or a regioisomeric mixture thereof, wherein R1-R6 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ -phenyl; or one of R₁ or R₂ and one of R₃ or R_{4;} or one of R₃ or R₄ and one of R₅ or R₆ form a fused ring, wherein the ring has 4-8 ring members; R₇-R10 are each, independently, hydrogen, hydroxyl, halogen, substituted or unsubstituted alkyl, substituted or unsubstituted alkoxy, nitro, cyano or amide; and n is an integer from 0 to 10.

In a preferred embodiment, R₁ and R₂ are alkyl, R₃-R₆ are, independently, H, OH, halogen, alkyl, alkoxy, substituted or unsubstituted acyl, substituted alkenyl or substituted alkyl carbonyl, and R₇-R₁₀ are hydrogen. In another preferred embodiment, R₁ and R₂ are each methyl and R₃-R₁₀ are each hydrogen. In 20 another preferred embodiment, R₁-R₄ are each hydrogen, R₅ and R₆ are each methyl and R₇-R₁₀ are each hydrogen.

Additional preferred β-lapachone analogs useful in the methods and kits of the present invention are recited in PCT International Application PCT/US03/37219 (WO2004/045557), which is incorporated by reference herein in its entirety, and which discloses compounds represented by Formula VIII: 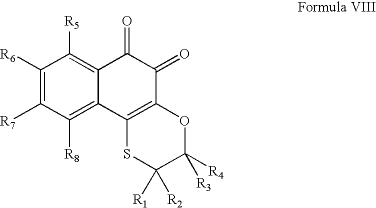
or pharmaceutically acceptable salts thereof, or a regioisomeric mixture thereof, wherein R₁-R₄ are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl; or one of R₁ or R₂ and one of R₃ or R4 form a fused ring, wherein the ring has 4-8 ring members; R₅-R₈ are each, independently, hydrogen, hydroxyl, halogen, substituted or unsubstituted alkyl, substituted or unsubstituted alkoxy, nitro, cyano or amide; and n is an integer from 0 to 10. In certain embodiments of Formula VIII, R₁, R₂, R₃, R_{4,} R₅, R_{6,} R₇ and R₈ are not each simultaneously H.

### 4. METHODS FOR DIAGNOSING, PREVENTING AND TREATING Mammalian Pre-Cancers, Cancers or Hyperproliferation Disorders

The present invention provides a method for preventing or treating mammalian pre-cancer, cancer, or hyperproliferation disorders. The method comprises activating Chk2 in the cells of the pre-cancer, cancer, or hyperproliferation disorders in the absence of de novo DNA damage. The Chk2 activators can be used as new drugs that kill cancer cells by increasing the activity of Chk2. The Chk2 activators are preferably not β-lapachone. The anticancer drugs promote cell death in pre-cancer or cancer cells, hyperproliferative cells or cells associated with DNA damage.

As described herein, Chk2 activation alone induced cell death both in a wide spectrum of cancer cell lines, and in a xenograft colon cancer model. In contrast, Chk2 activation in the absence of DNA damage exerted little effect on the non-transformed cells. Thus, the Chk2 activators of the present invention are drug candidates for the prevention and treatment for pre-cancers, cancers, or hyperproliferation disorders.

4.1. The Pre-cancers, Cancers, or Hyperproliferation Disorders

Various cancers to be treated include but are not limited to lung cancer, colorectal cancer, breast cancer, pancreatic cancer, ovarian cancer, prostate cancer, renal carcinoma, hepatoma, brain cancer, melanoma, multiple myeloma, hematologic tumor, and lymphoid tumor. Hyperproliferative disorders refer to conditions in which the unregulated and/or abnormal growth of cells can lead to the development of an unwanted condition or disease, which can be cancerous or non-cancerous, for example a psoriatic condition. As used herein, the term “psoriatic condition” refers to disorders involving keratinocyte hyperproliferation, inflammatory cell infiltration, and cytokine alteration. Hyperproliferative diseases/disorders to be treated include but are not limited to epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses and the like. The Chk2 related disorder can be a DNA repair disorder, including but not limited to, Ataxia-Talangiectasia, premature aging syndrome, Li-Fraumeini syndrome and premalignant conditions, such as BRCA families. The compositions of the present invention may also be useful for pre-cancer, clinical conditions that bear increased risk of progression into cancer.

In an embodiment, the cancer is p53-deficient cancer. It has been proposed that a primary function of Chk2 is to mediate activation of p53 and pro-apoptotic p53 target genes following DNA damage (Takai et al.

Embo J 21:5195-205 (2002); Hirao et al.

Mol Cell Biol 22:6521-32 (2002)). However, the Chk2-p53 pathway is compromised in the majority of human cancers due to frequent inactivation of p53. In cancerous stage, Chk2 activity was down regulated and its anti-cancer activity was compromised due to p53 inactivation.

The present invention demonstrates that activation of a p53-independent Chk2 response pathway force cancer cells to respond to checkpoint activation. As described herein, activation of Chk2 leads to both senescence and apoptosis in cancer cells without functional p53. Thus, the induced activation of Chk2 uncovers and enhances functions of p53-independent anti-cancer pathways of Chk2, which is subdued due to p53 inactivation in cancer cells.

In another embodiment, the cancer is a cancer with increased survivin level. Potent anti-apoptotic pathways allow cancer cells to evade cell death and to continue aberrant proliferation. For example, survivin, an inhibitor of apoptosis protein (IAP), has been found to be required for cancer cell survival in the majority of human cancers. Increased survivin expression has been detected in the majority of human tumor types, which constitutes a major anti-apoptosis mechanism in human cancer. Inhibition of survivin alone through siRNA, antisense oligonucleotides, chemical inhibitor, or dominant-negative mutants is sufficient to induce selective cancer cell death (Lu, et al., Cancer Res, 64:2840-2845 (2004); Coma, et al., Oligonucleotides, 14:100-113 (2004); Reed, J Clin Invest, 108:965-969 (2001)) in a broad spectrum of malignant cells.

The present invention demonstrates that Chk2 activation induces comparable, if not more potent, ablation of survivin overexpression prior to selective cancer cell killings. As described herein, the cancer cell death due to Chk2 activation is found to correlate with a reduction in survivin protein levels. In addition, early stage breast cancer samples demonstrate an inverse correlation between Chk2 activation and survivin protein levels.

4.2. Cell Death of Cancer Cells

The present invention is also broadly drawn to the use of the candidate compounds to modulate cell death of cancer cells. Modulation of apoptosis using these test compounds can occur in vitro, in vivo, or ex vivo. Modulation can occur in cancer cells, cell lines, and primary cells.

Another embodiment of the present invention is a method for preventing or inhibiting growth of pre-cancer or cancer cells, the method comprising administering to the cells a composition, which enhances Chk2 activity or expression in the cell in an amount sufficient to inhibit growth of pre-cancer or cancer cells. The method can be carried out on mammalian cells, including human cells, and can be carried out in vitro or in vivo.

Another embodiment of the present invention is a method for diagnosing and treating mammalian pre-cancer or cancer in a subject, the method comprising obtaining pre-cancer or cancer cells from the subject; testing the pre-cancer or cancer cells from the subject for the presence of Chk2; and administering to the subject a composition which enhances Chk2 activity or expression, in an amount sufficient to inhibit growth of pre-cancer or cancer cells. The composition used in this method can be a β-lapachone analog, derivative, or metabolite thereof, or an mRNA, which increases the expression of Chk2.

Another embodiment of the present invention is a method for treating pre-cancer or cancer in a mammalian subject, the method comprising administering to the mammal a composition which enhances Chk2 activity or expression and monitoring the mammal to determine the state of the pre-cancer or cancer, wherein the composition is administered in an amount sufficient to inhibit the growth of pre-cancer or cancer cells. The composition used in this method can be a β-lapachone analog, derivative, or metabolite thereof, or an mRNA, which increases the expression of Chk2.

Another embodiment of the present invention is a method for diagnosing patients who would be receptive to treatment with a composition, which, when administered to mammalian subjects with pre-cancer or cancer, selectively enhances activation or expression of Chk2 and results in regression of tumor cell growth in the mammalian subjects. The method comprises obtaining cells from the patient; testing the cells for the presence of either Chk2 or for the presence of Chk2 activity; wherein the presence in the cells of either Chk2 or for the presence of Chk2 activity indicates a patient who would be receptive to treatment.

In this embodiment, compounds such as β-lapachone analogs, derivatives or metabolites thereof could be used to diagnose cancer. Cells isolated from subjects could be cultured in the presence or absence of β-lapachone analogs, derivatives or metabolites thereof. Cells that have their growth rates inhibited in the β-lapachone analogs, derivatives or metabolites thereof treated cells relative to control would be pre-cancer or cancer cells. The subject could then be diagnosed with pre-cancer or cancer.

The Chk2 activators of the present invention may help the development of drugs that inhibit apoptosis during various forms of tissue injury such as ischemia, reperfusion injury, mechanical injury, inflammation or immunological damage.

4.3 The Compositions for the Test Compounds and Chk2 Activators

As discussed above, in one aspect, the present invention provides a composition, which, when administered to mammalian subjects with pre-cancer, cancer or a hyper-proliferative disorder, selectively enhances Chk2 activity and results in regression of cell growth in mammalian cells and subjects. The composition can also be in the form of a pharmaceutical composition or in a kit.

The compositions of the present invention can be incorporated into pharmaceutical compositions suitable for administration. Such compositions typically comprise the substance that enhances Chk2 activity or expression and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the present invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., the substance that enhances Chk2 activity or expression) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### EXAMPLES

Examples are provided below to further illustrate different features of the present invention. The examples also illustrate useful methodology for practicing the invention. These examples do not limit the claimed invention.

### Example 1

### Methods

### 1. Cell Culture and Expression Vectors

IMR-90, A549, NCI-H460, NCI-H1299, 293, DLD1, HT-29, and HCT-116 cells were maintained in Dulbecco's Modified Eagle Medium (DMEM) supplemented with 10% fetal bovine serum (FBS) and 1% penicillin/streptomycin (PS) (Roche). NCM-460 cells were maintained in M3D media (Cambrex) supplemented with 10% FBS and 1% PS. The Chk2-inducible cell line was maintained in DMEM supplemented with 10 μg/ml Blasticidin and 500 μg/ml Zeocin. Chk2 induction was performed by addition of 1000 ng/ml tetracycline to growth medium unless specified otherwise. To create tetracycline-inducible Chk2 expression vector pcDNA4/TO-Chk2, the coding region of Chk2 was amplified from full length Chk2 cDNA clone (Invitrogen) by polymerase chain reaction (PCR) and inserted into the HindIII/EcoRI sites of pcDNA4/TO (Invitrogen). The DLD1-T-Rex cell lines were generated by stably transfecting the DLD1 cancer cell lines with the regulatory vector pcDNA6/TR (Invitrogen) under selection with 10 μg/ml Blasticidin. Chk2-inducible cell lines were generated by stably transfecting the DLD1-T-Rex with the expression vector pcDNA4/TO-Chk2 under selection with 500 μg/ml Zeocin.

### 2. Adenoviral Vectors

Recombinant adenoviral vectors encoding chk2 (AdChk2) and daxx (AdDaxx) were created by homologous recombination in Escherichia coli as described by the manufacturer (Stratagene). Control (AdCon) virus contains an empty expression cassette and had no effect on parameters tested relative to mock infection (data not shown). Viruses were propagated in 293 cells (Stratagene), purified by centrifugation through cesium chloride gradients, and titers determined by immunohistochemical staining for the adenovirus hexon protein as described previously (Rogoff et al., Mol Cell Biol 22, 5308-18, 2002). Viral infections were carried out at 37° C. in 5% carbon dioxide for 1 hour as described previously (Rogoff et al., Mol Cell Biol 22, 5308-18, 2002).

### 3. Apoptosis Analysis

For flow cytometry analysis, cells were plated at 6000 cells per cm² 24 hours prior to infection with AdCon or AdChk2. Cells were harvested at 48 hours post infection (hpi) and stained with propidium iodide (PI) as described previously (Yang, et al., Nat Cell Biol 4.865-870 (2002)). Cells undergoing apoptosis were defined as the population that contains less than a G1 DNA content. For MTT analysis, cells were plated in 6 well dishes and transfected with siRNA oligonucleotides at 24 hours after plating. Cells were then split into 12 well plates at 2×10⁴ cells per well. At 24 hours following transfection, cells were infected with recombinant adenoviral vectors or treated with indicated compound. At 48 hours after treatment, MTT was added to each well (0.5 mg/ml, final concentration) and plates were incubated for 2 h at 37° C. Medium was aspirated and formazan product was solubilized in 250 μl of isopropyl alcohol. The absorbance of each well was measured at 570 nm using a microplate reader.

### 4. Analysis of Senescence Senescence was assessed by staining for senescence-associated μ-galactosidase activity as suggested by the manufacturer (Senescence Staining Kit, Cell Signaling).

### 5. Mitochondrial Potential

Changes in mitochondrial membrane potential were monitored using DePsipher Dye (Trevigen). This dye forms red aggregates upon membrane polarization and reverts to its green monomeric form if the potential is disturbed. The red aggregates have absorption/emission maxima of 585/590 nm and the green monomers of 510/527 nm. Briefly, DLD1-tet-Chk2#17 cells were pretreated with tetracycline for 7 days and were incubated with 10 μM DePsipher at 37 ° C. for 15 minutes, washed twice, and resuspended in phosphate-buffered saline. Images with a fluorescein filter and rhodamine filter were taken with a Nikon Eclipse TE300 microscope.

### 6. Immunofluorescence

Cells were plated at 1×104 cells per well in 8 well chamber slides (Nalge Nunc International). At 24 hours post plating, cells were coinfected with AdDaxx at an MOI of 10 and either AdCon or AdChk2 at an MOI of 25. At 24 hpi, cells were fixed in 4% parafonnaldehyde and permeablized with 0.2% Triton X-100. Cells were then blocked in 1% BSA followed by incubation with anti-Daxx polyclonal antisera (Santa Cruz Biotechnology). Bound antibodies were detected using a fluorescein isothiocyanate (FITC) conjugated secondary antibody (Jackson Immunoresearch).

### 7. Immunoblotting

Cultured cells were harvested and lysed in whole-cell extract buffer (50 mM Tris-HcL pH 7.5, 150 mM NaCl, 1.0% NP-40, 1 mM EDTA, 0.1 mM sodium orthovanadate, 1× protease inhibitor cocktail (Roche)) by incubation for 30 min on ice. Soluble proteins were separated by centrifugation at 13,000×g in a microcentrifuge, and supernatants were stored at −70° C. Proteins were separated by sodium dodecyl sulfate-polyacrylamide gel electrophoresis analysis and transferred to a polyvinylidene difluoride membrane (Biorad) by electroblotting. Chk2 was detected with Chk2 monoclonal antibody clone #7 (Labvision), the phospho-threonine 68 form of Chk2 was detected with polyclonal antisera specific for that modification (Cell Signaling), Daxx was detected with polyclonal antisera M-112 (Santa Cruz), Ask1 was detected with monoclonal antibody clone F-9 (Santa Cruz). Following secondary antibody incubation (Amersham), bound antibodies were detected by chemiluminescence (Pierce).

8. siRNA

Chemically synthesized Smart Pool siRNA molecules targeting Chk2, Daxx, Ask1, as well as the non-targeting control siRNA were generated by Dharmacon, Lafayette, Colo. siRNA oligonucleotides were transfected into cells at a concentration of 100 nm using Lipofectamine 2000 (Invitrogen) as described by the manufacturer.

9. Xenograft tumor growth inhibition.

Female 4- to 5-week-old nude mice (Charles River Laboratories, Wilmington, Mass.) were maintained in the animal facility at the ArQule Biomedical Institute. Mice were divided into four experimental groups with five mice per group. DLD1-Control and DLD1-Chk2 cells (4×10⁶ cells) were injected subcutaneously into the flanks of mice. After cell inoculation, animals were fed drinking water containing 5% sucrose in the presence or absence of 1 mg of Tetracycline per ml (Sigma). Drinking water was changed every other day for a 5-week period of tumor formation. Tumor volumes were measured and recorded two times a week from day 4 of cell inoculation. At the end of 5 weeks, the mice were sacrificed, and the tumors were removed for detection of Chk2 expression. Data were repeated twice in two independent experiments. Error bars represent standard deviations of tumor volumes of the five mice in each group. P value of 0.019 represents significance of tumor growth inhibition between DLD1-Chk2 and DLD1 parental cell line derived tumors with the addition of tetracycline.

10. Immunohistochemsitry

Xenograft cancer tissues were dissected and fixed in 10% PBS buffered formaldehyde for 48 hr and then processed for 4 μM section slides. Early stage breast cancer samples were purchased from Asterand, Detroit, Mich., and then processed for 4μM section slides. The slides were boiled in 10 mM citric acid (pH 6.0) for 10 min prior to incubation overnight at 4° C. with phospho-threonine 68 Chk2 polyclonal antisera (Cell Signaling) or survivin polyclonal antisera (Lab Vision). Staining was visualized with DAB after application of super-sensitive IHC system as directed by the manufacturer (Biogenex).

### Example 2

### Increased Expression of Chk2 Leads to Activation of Kinase Activity.

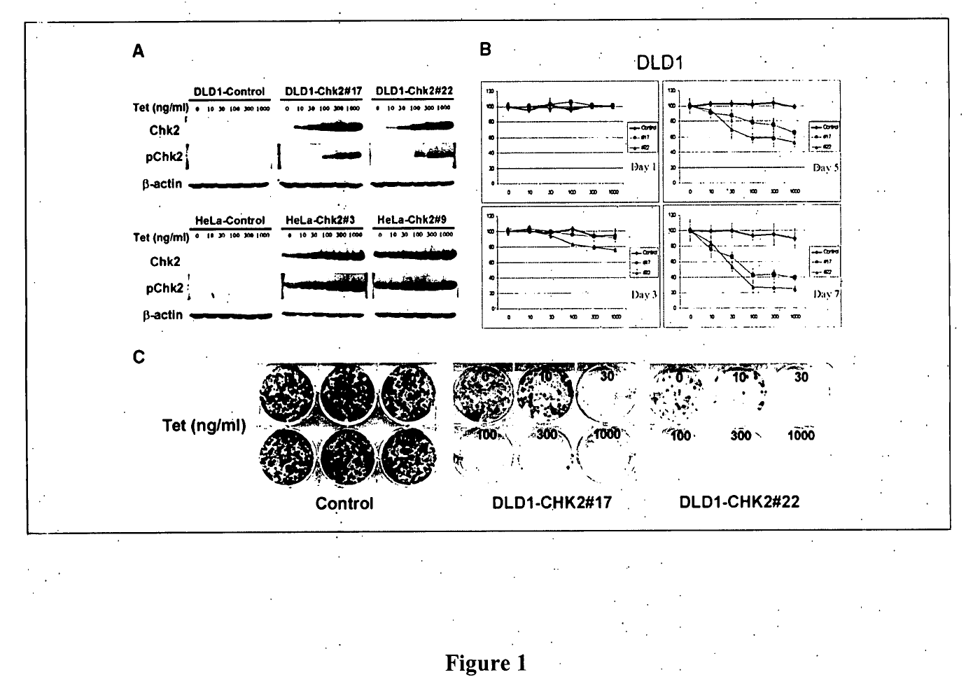

To study the effects of Chk2 expression in p53-deficient cancer cells, a tetracycline-inducible (tet-on) Chk2 expression system was generated in DLD1 and HeLa cancer cell lines, which harbor mutant p53 (DLD1) and undetectable wild-type (wt) p53 due to E6-mediated degradation (HeLa). Several DLD1-Chk2 and HeLa-Chk2 clones were selected that behaved similarly in all the assays performed. Two representative clones (#17 and #22 for DLD1 cells; #3 and #9 for HeLa cells) were chosen from each cancer cell line for further analyses. Western blot analysis demonstrated that addition of tetracycline to the culture medium resulted in Chk2 protein production in a dosage-dependent manner in DLD1-Chk2 and HeLa-Chk2 cell lines (FIG. 1A). Cell lines with vector alone did not induce Chk2 protein production in the presence of tetracycline (FIG. 1A). Next, the phosphorylation status of Chk2 was examined and it was shown that Chk2 protein could undergo phosphorylation at its activation site, threonine 68 (FIG. 1A). In contrast, phosphorylation of threonine 68 on Chk2 was not detected in uninduced or control cells. Thus, Chk2 protein expression is tightly controlled in the DLD1-Chk2 and HeLa-Chk2 tet-on systems.

### Example 3

### Chk2 Expression Inhibited Cell Growth

In order to understand the biological effects of Chk2 expression, the effect of Chk2 expression on cell proliferation was examined. Increasing concentrations of tetracycline were added to the culture medium of the DLD1-Control and DLD1 -Chk2 (clone#17 and #22) cells and MTT and colony formation assays were performed. Induction of Chk2 expression was found to suppress cell proliferation in both MTT assays and colony formation assays (FIG. 1B and 1C). Similar results were obtained in HeLa-Chk2 cell lines. In contrast, growth of DLD1-Control cells was not affected by tetracycline addition (FIG. 1B and 1C). These results indicate that Chk2 expression induces cell cycle arrest and/or cell death.

### Example 4

### Chk2 Activation Leads to G2 Arrest and Cell Death

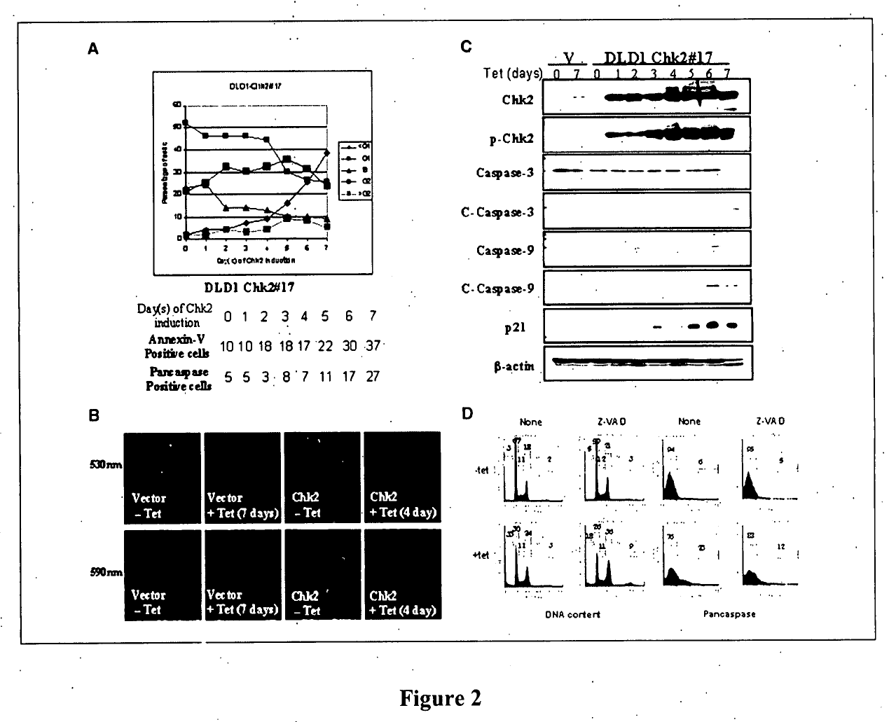

To investigate the effect of Chk2 expression on cell cycle progression, the cell cycle profiles of DLD1-Chk2 clones (#17 and #22) were analyzed after Chk2 induction by addition of tetracycline. Results show that Chk2 expression resulted in an increase in cell populations with sub-G1 DNA content (˜4% to ˜40%) and G2/M DNA content (20% to 30-35%) and a decrease in populations with G1 DNA content (˜52% to ˜25%) and S DNA content (˜25% to ˜9%), compared with uninduced culture or control cells (FIG. 2A). These results indicate that Chk2 expression can result in both a G2/M delay and cell death. To further analyze the Chk2-induced cell cycle arrest and determine whether it was G2/M transition arrest or mitotic arrest, tetracycline-treated DLD1-Chk2 cells were stained with an anti-phospho-Histone H3 serine 10 antibody that identifies mitotic cells. The results showed no increase in phospho-Histone H3 in tetracycline-treated DLD1-Chk2 cells. Taken together, these results indicate that expression of Chk2 resulted in G2 arrest and cell death.

### Example 5

### Chk2 Expression Induces Apoptosis After G2/M Phase Arrest

To distinguish apoptosis and necrosis, tetracycline-treated DLD1-Chk2 cells were subjected to Annexin-V staining and Pancaspase assay. Results showed that the numbers of Annexin-V-positive and Pancaspase-positive cells were increased significantly after 2 to 3 days of Chk2 induction (FIG. 2A). In addition, an increase in the active form of caspase-3 and caspase-9 and a decrease in the mitochondrial potential were observed in cells expressing Chk2 (FIG. 2 C). Furthermore, Chk2-induced cell death was partially rescued by Z-VAD, a general caspase inhibitor (FIG. 2D). Similar results were obtained in HeLa-Chk2 cells. In contrast, no increase of Annexin-V-positive or Pancaspase-positive cells, active caspase-3 and -9, or decrease in mitochondrial potential were observed in uninduced or control cells (FIG. 2A-2C).

Collectively, these results indicate that expression of Chk2 resulted in caspase activation and apoptosis.

### Example 6

### Chk2 Expression Induces Cellular Senescence

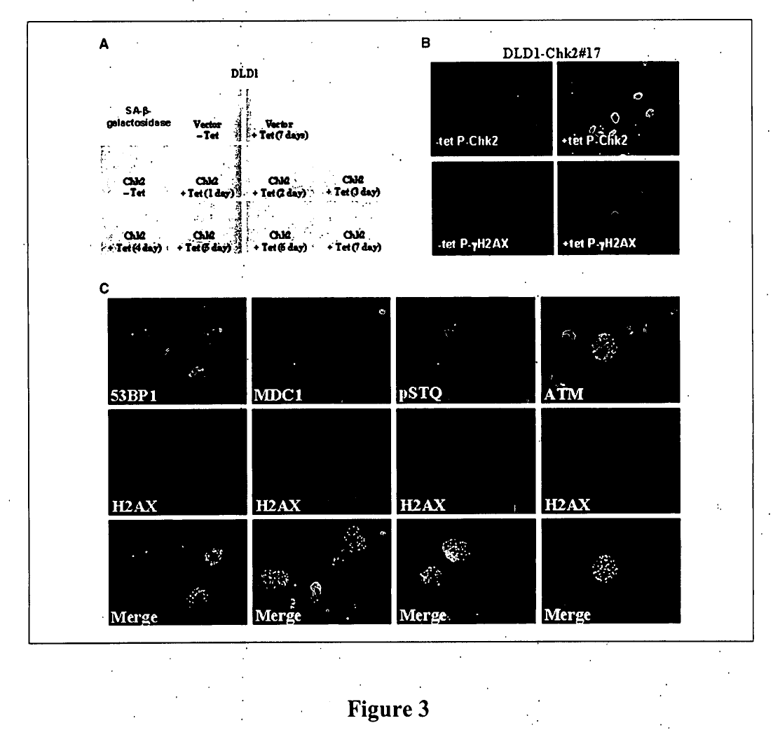

In addition to cell cycle arrest and apoptosis, expression of Chk2 resulted in cells assuming a large and flat morphology, with abundant cytoplasmic vacuolization. Similar phenotypic changes have been frequently observed in senescent cells. To assess whether DLD1 cells undergo cellular senescence following Chk2 expression, the activity of the senescence-associated β-galactosidase (SA-β-gal), a specific marker of cellular senescence, was analyzed. Approximately 15% of DLD1-Chk2 cells were positive for SA-β-gal staining after 4 days of Chk2 induction, and the percentage was increased further to approximately 35% after 7 days of induction (FIG. 3A). In addition, Chk2-expressing cells had increased p21 expression (FIG. 2C), which is frequently found in senescent cells. Similar results were obtained following expression of Chk2 in HeLa cells. These data indicate that expression of Chk2 can result in cellular senescence.

### Example 7

### Chk2-expressing Cells Form Nuclear Foci

The accumulation of γ-H2AX foci has been found in both apoptotic and senescent human cells (Rogakou et al., J Biol Chem. 275: 9390-5, 2000; Sedelnikova et al., Nat Cell Biol. 6:168-70., 2004). γ-H2AX foci reveal DNA double-strand breaks (DSBs) and co-localize with DSB repair factors such as ATM, p53BP1, and MDC1. To investigate whether γ-H2AX foci were present in cells following expression of Chk2, the incidence of foci in DLD1-Chk2 cells was examined by immunofluorescence. The appearance of γ-H2AX in 10-15% of DLD1-Chk2 cells was found after three days of Chk2 induction, and the percentage increased to 25-30% after 7 days of Chk2 induction (FIG. 3B). In addition, co-localization of ATM, 53BP1, and MDC1 with the γ-H2AX foci was also found (FIG. 3C). Similar results were observed following expression of Chk2 in HeLa cells.

### Example 8

### Increased Expression of Chk2 Leads to Chk2 Activation.

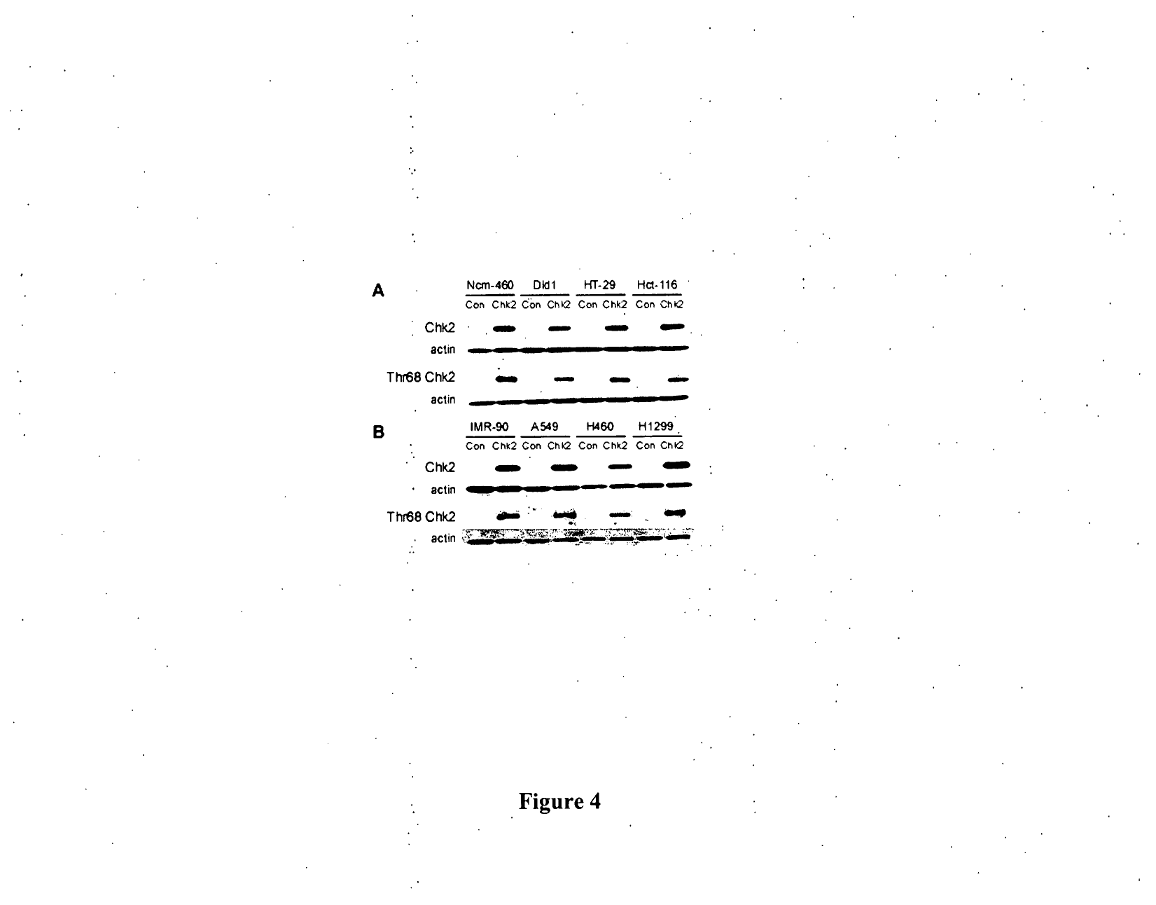

To determine the response of cancer cells to direct activation of the Chk2-mediated checkpoint pathway, an adenoviral gene transduction was used to overexpress Chk2 in cancer and non-transformed cell lines. Chk2 was expressed ectopically in human cancer cell lines, including colon cancer cell lines Dld1, HT-29, and HCT-1 16, and lung cancer cell lines A549, NCI-H460, and NCI-H1299, and melanoma, pancreatic cancer, ovarian cancer, and prostate cancer cell lines. Chk2 was also ectopically expressed in immortalized, non-transformed cell lines, including NCM-460 and IMR-90 cell lines. The cell lines were infected with AdChk2 at an multiplicity of infection (MOI) of 25. Cells were harvested and extracts made at 24 hours post infection (hpi). Actin is shown as a protein loading control. (FIG. 4A).

The activation status of over expressed Chk2 was then determined in the different cell lines, by detecting levels of Chk2 phosphorylated at the threonine 68 residue (Melchionna, et al., Nat Cell Biol 2:762-5 (2000); Ahn, et al., Cancer Res 60:5934-6 (2000)). Using an antibody specific for a phosphorylated form of Chk2, over expression of Chk2 alone was found to be sufficient for activation of Chk2 as measured by the increase in the phospho-threonine 68 form of Chk2 (FIG. 4B). These data demonstrate that overexpression of Chk2 is sufficient to result in Chk2 activation, likely through Chk2 autophosphorylation.

### Example 9

### Increased Activation of a Chk2 Induces p53-independent Apoptosis in Tumor Cells.

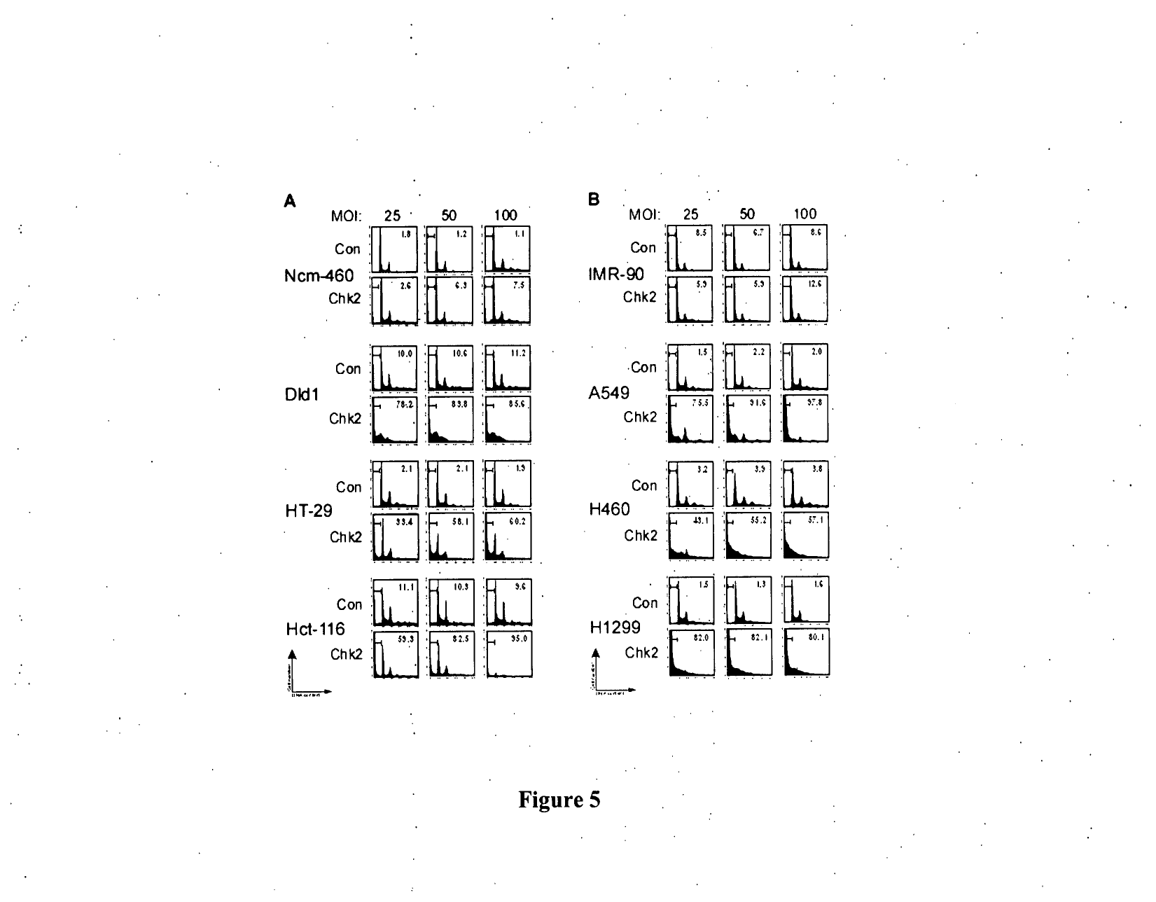

It was next determined if activation of checkpoint kinase alone was sufficient to induce apoptosis in cancer cells in the absence of de novo DNA damages. Cell lines were infected with AdCon or AdChk2 at the indicated MOI. Cells were harvested and DNA stained with propidium iodide (PI) at 48 hpi. Number in each panel represents percentage of cells with sub-G1 DNA content. Chk2 activation alone potently induced apoptosis in a wide spectrum of cancer cell lines, including three colon cancer cell lines (FIG. 5A) and three lung cancer cell lines (FIG. 5B) as measured by sub-2N DNA content. Additionally, expression of Chk2 was found to induce apoptosis in breast, melanoma, renal, and pancreatic cancer cell lines (data not shown). Cancer cells lacking functional p53 were as susceptible to apoptosis induced by over expression of Chk2 as were cells with wild type p53, suggesting that Chk2 induces apoptosis by both p53-dependent and p53-independent mechanisms. In contrast, Chk2 activation in non-tumorogenic, early passage NCM-460 colon cells and IMR-90 lung cell lines showed little or no increase in apoptosis over control virus infected cells (FIGS. 5A and 5B). Taken together, these data show that activation of a Chk2 selectively induces apoptosis in tumor cells regardless of p53 status.

### Example 10

### Role of Survivin in Chk2-induced Cell Death

We next determined the p53 independent mediator of selective cancer cell apoptosis induced by Chk2. Cancer cells have constitutive propensity to apoptosis due to genomic instability, constitutive oncogene activation, and other aberrations. Survivin was tested to determine if it was involved in the selective cancer cell killing by Chk2 activation.

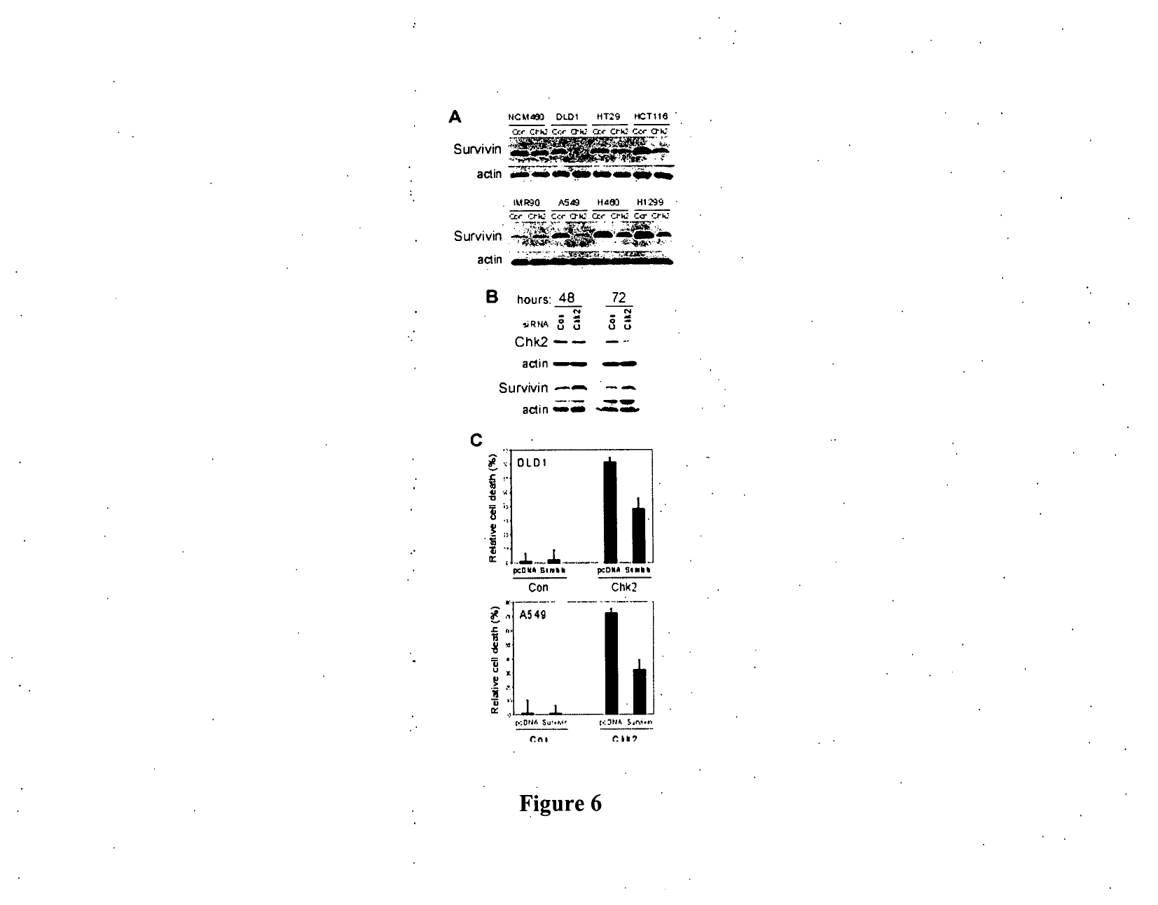

It was found that ectopic expression of Chk2 resulted in potent ablation or normalization of survivin overexpression (FIG. 6A). The survivin down regulation by Chk2 activation is comparable to, if not more potent than, those induced by siRNA or chemical inhibitors reported (Kappler, et al, Cancer Gene Ther, 11:186-193 (2004); Ning, et al, Int JOncol 25:1065-1071 (2004); Wu, et al, J Biol Chem 280:9745-9751 (2005); Wall, et al, Cancer Res 63:230-235 (2003)). The normalization or reduction of survivin expression by Chk2 occurs prior to the detection of cell death, which suggests that down regulation of survivin overexpression is not a result of, but likely to be the mechanism of cancer cell death.

The potent reduction of survivin expression upon Chk2 activation suggests Chk2 may function as an endogenous regulator of survivin protein levels. To investigate this interaction between survivin and Chk2 under non-overexpressed conditions, we undertook a reverse approach by silencing chk2 with specific siRNA. Endogenous Chk2 expression was reduced by siRNA targeting chk2 in cells (FIG. 6B). We found that a reduction in Chk2 protein levels led to an increase in survivin protein levels (FIG. 6B and data not shown). This result further supports Chk2 as a negative regulator of survivin under both over- and non-overexpressed conditions.

It was next determined if survivin reduction contributed to Chk2-induced apoptosis in cancer cells. Co-expression of survivin with Chk2 has proven difficult due to the potent negative effects of Chk2 on survivin expression as described above. DLD1 colon cancer cells or A549 lung cancer cells were transfected with survivin prior to infection with AdChk2. Moderately increased survivin expression was achieved, which led to a decrease in apoptosis as compared to expression of Chk2 alone (FIG. 6C). Taken together, these data demonstrate that Chk2 negatively regulates survivin protein levels, and that the decrease in survivin levels contributes to Chk2-induced apoptosis.

Normal tissues express little or very low level of survivin under non-stressed conditions. Non-transformed and immortalized cells express lower level of survivin than transformed cells. To our surprise, Chk2 activation induced little or no change in survivin protein expression in the non-transformed cell lines (FIG. 6A). These results suggest that the down regulation of survivin protein levels by Chk2 is through a survivin upregulation mechanism that is important in malignant cells.

### Example 11

### Chk2 Activation Inhibits Tumor Growth and Survivin Expression in Vivo

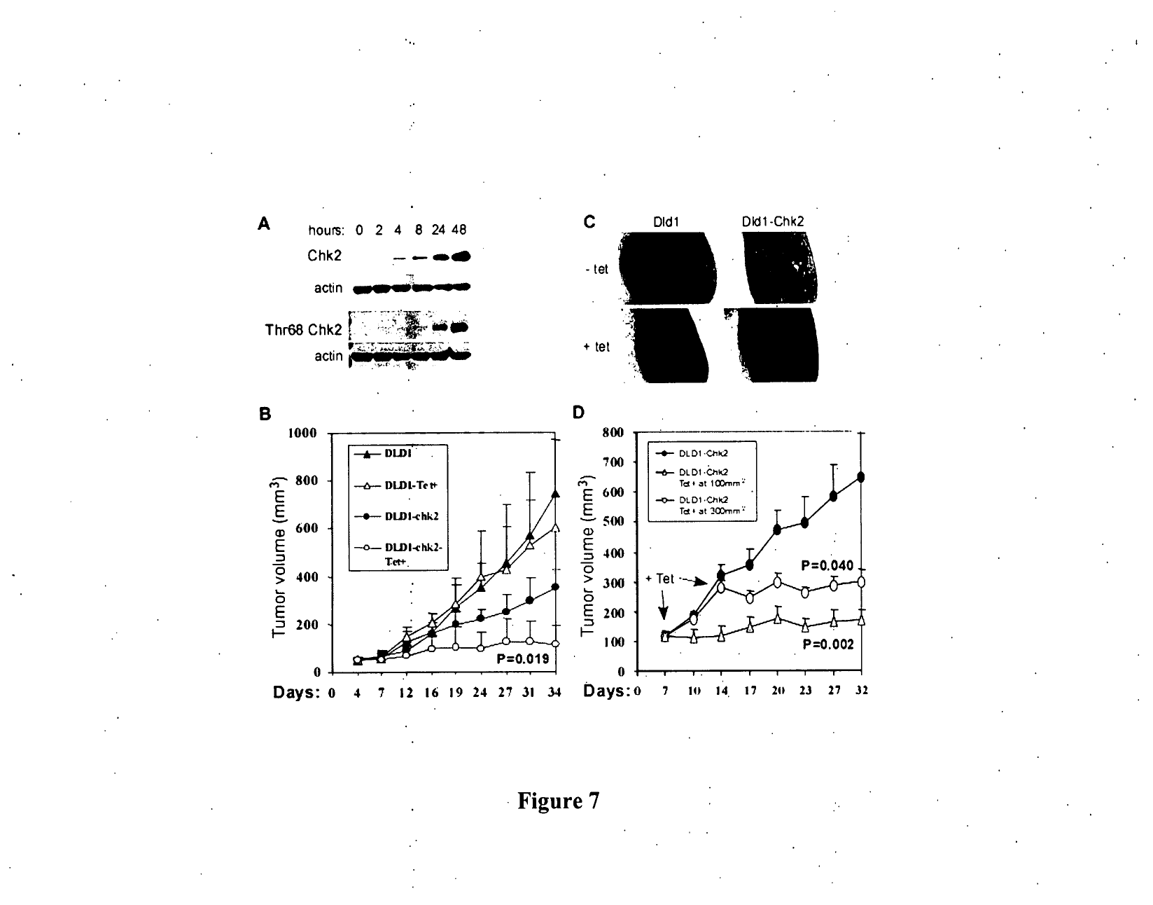

We next determined the effect of Chk2 activation on tumor growth in vivo. Human DLD1 colon cancer cells were used to generate a tetracycline-inducible Chk2 cell line (Chen, et al., Cancer Res, 65.-6017-6021 (2005)). Addition of tetracycline to the growth medium of DLD1-Chk2 cells in culture resulted in a rapid activation of Chk2 as measured by an increase in the phospho-threonine 68 form of Chk2 (FIG. 7A). Chk2-inducible DLD1 cells and the vector control parental cells were implanted under the skin of nude mice, and expression of chk2 was triggered by addition of tetracycline to the drinking water. The induction of Chk2 resulted in potent anti-tumor activity that nearly completely suppressed the growth of xenografted human colon cancer (P=0.019), while tumors derived from the DLD1 parental cell line continued to grow throughout the duration of the experiment (FIG. 7B, 7C). Interestingly, tumors derived from DLD1 -Chk2 cells also showed trend of retarded growth in the absence of tetracycline. On further examination, this line showed high basal level of “leaking” Chk2 expression in the absence of tetracycline (data not shown). We also determined the effect of Chk2 activation on the growth of established tumors. Chk2-inducible DLD 1 cells were implanted under the skin of nude mice, and tumors allowed to develop to 100 mm³ or 300 mm³. Following tumor establishment, expression of chk2 was triggered by addition of tetracycline to the drinking water. The induction of Chk2 resulted in potent anti-tumor activity that nearly completely suppressed the growth of the xenografted human colon cancer, while tumors lacking tetracycline addition continued to grow throughout the duration of the experiment (FIG. 7D). These data demonstrate that Chk2 activation alone has potent anti-tumor activity in vivo in the absence of de novo DNA damage.

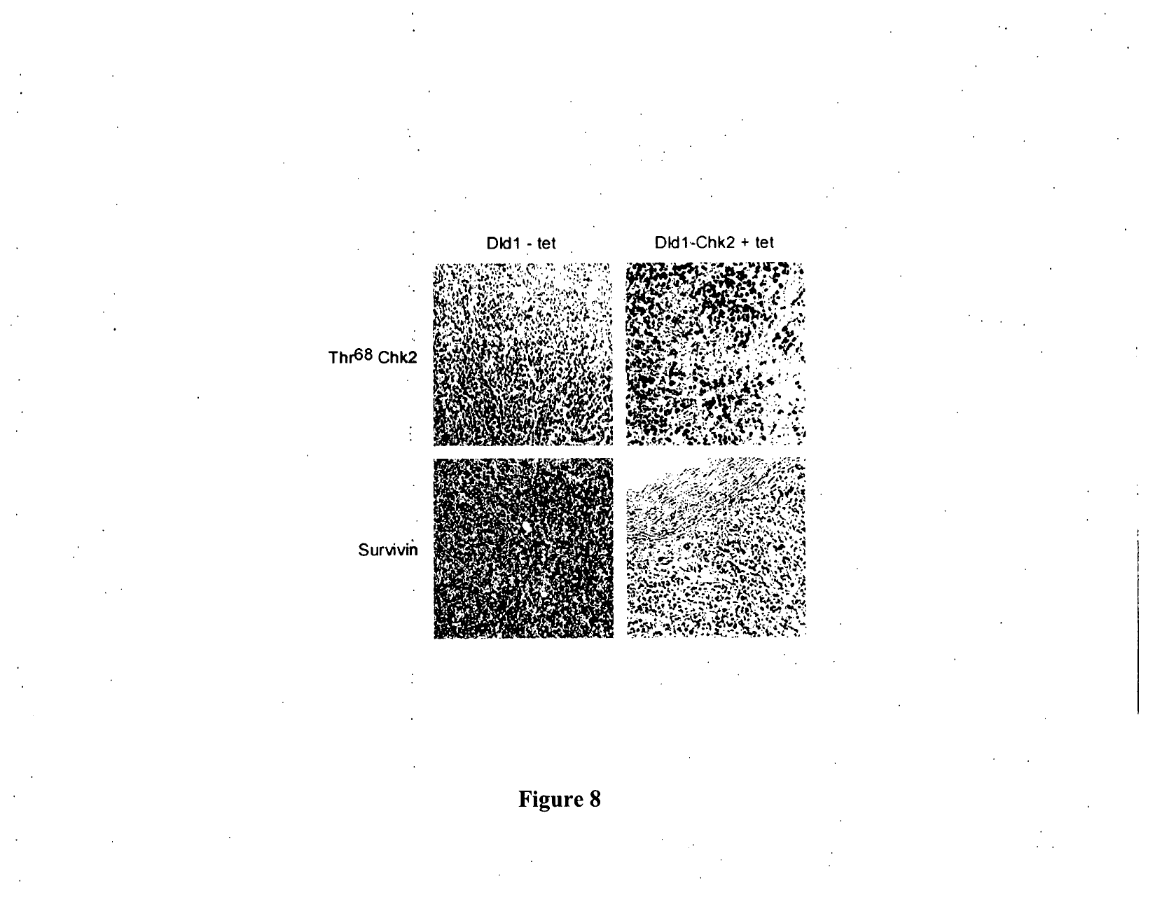

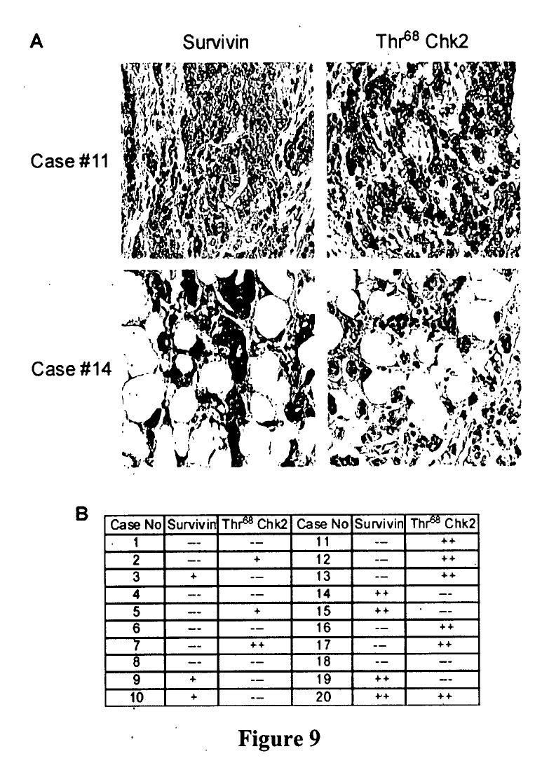

We next determined whether expression of Chk2 in vivo resulted in down regulation of survivin similar to the reduction observed in cell culture. Xenograft human colon tumors derived from DLD1 or DLD1-Chk2 cells were inmunohistochemically stained for expression of survivin and the phospho-threonine 68 form of Chk2. We observed an increase in the phospho-threonine 68 form of Chk2 in the DLD1-Chk2 tumors following administration of tetracycline to the animals (FIG. 8). Consistent with our in vitro observations (FIG. 6), we observed a reduction in survivin protein levels in the DLD1 -Chk2 derived tumors as compared to tumors derived from the DLD1 cell line (FIG. 8).

We next wanted to examine the correlation between survivin and Chk2 activity in human tumor samples. While DNA damage checkpoints are found to act as an anti-cancer barrier in early human carcinogenesis, this endogenous defense against malignant transformation is compromised at advanced stages of tumorigenesis (Gorgoulis, et al, Nature 434:907-913 (2005); Bartkova, et al, Nature 434:864-870 (2005)). We therefore examined early stage breast cancer samples for expression of survivin and phosphorylated Chk2. In 14 out of the 20 cases analyzed, survivin levels showed a negative correlation to levels of the phospho-threonine 68 form of Chk2 (FIG. 9A, 9B). Taken together, these data suggest that activation of the Chk2 in vivo results in degradation of survivin early in tumorogenesis, and suggests that inactivation of this checkpoint pathway allows for increased survivin expression in later stages of tumor formation.

Other embodiments are within the following claims. While several embodiments have been shown and described, various modifications may be made without departing from the spirit and scope of the present invention.

What is claimed is:

1. A method for screening for an activator of Chk2 comprising the step of assessing the Chk2-activating effect of a test compound in cells containing DNA encoding Chk2.

2. The method of claim 1 wherein the step of assessing the Chk2-activating effect in cells comprising,
exposing the cells to a test compound,
measuring the activity of Chk2 in the cells in the presence and in the absence of the test compound, and
comparing the activity of Chk2 in the presence and in the absence of the test compound.

3. The method of claim 1 wherein Chk2-activating effect is determined by an increase in the phosporylation of Chk2.

4. The method of claim 1 wherein the cells are cancer cells.

5. The method of claim 4 wherein the cancer cells are cells in a cancer of a vertebrate, mammal, or human.

6. The method of claim 4 wherein the cancer cells are derived from a cancer of a vertebrate, mammal, or human.

7. The method of claim 4 wherein the cancer cells are cultured cancer cells.

8. The method of claim 7 wherein the cultured cancer cells are selected from the group consisting of A549, NCI-H460, NCI-H1299, DLD1, HT-29, and HCT-116 cells.

9. The method of claim 1 wherein the test compound is a small molecule.

10. The method of claim 1 wherein the test compound is an analog, derivative, or metabolite of β-lapachone.

11. The method of claim 6 further comprising the step of assessing the Chk2-activating effect of the test compound in normal cells containing DNA encoding Chk2.

12. The method of claim 11 wherein the step of assessing the Chk2-activating effect in normal cells comprising,
exposing the normal cells to a test compound,
measuring the activity of Chk2 in the normal cells in the presence and in the absence of the test compound, and
comparing the activity of Chk2 in the presence and in the absence of the test compound.

13. The method of claim 11 wherein the normal cells are normal cells in a vertebrate, mammal, or human.

14. The method of claim 11 wherein the normal cells are normal cells derived from a vertebrate, mammal, or human.

15. The method of claim 11 wherein the normal cells are cultured normal cells.

16. The method of claim 15 wherein the cultured normal cells are selected from the group consisting of IMR-90, MCF-10A, NCM460, and PBMC cells.

17. The method of claim 11 further comprising the step of selecting the test compound that has a higher Chk2-activating effect in the cancer cells than in the normal cells.

18. A method for screening for a Chk2 activator, comprising the step of assessing the Chk2-activating of a test compound in the lysate of cells containing DNA encoding Chk2.

19. The method of claim 18 wherein the cells are cancer cells.

20. The method of claim 19 further comprising,
assessing the Chk2-activating effect of the test compound in the lysate of normal cells containing DNA encoding Chk2, and
comparing the Chk2-activating effects of the test compound in the cancer cell lysate and the normal cell lysate.

21. A method for screening for a Chk2 activator comprising,
contacting Chk2 with a test compound,
measuring the activity of Chk2 in the presence and in the absence of the test compound, and
comparing the activity of Chk2 in the presence and in the absence of the test compound.

22. The method of claim 21 further comprising selecting the test compound that increases the Chk2 activity.

23. The method of claim 22 further comprising,
assessing the Chk2-activating effect of the selected compound in cancer cells containing DNA encoding Chk2, or the lysate of the cells,
assessing the Chk2-activating effect of the selected compound in the lysate of normal cells containing DNA encoding Chk2, or the lysate, and
comparing the Chk2-activating effects of the selected compound in the cancer cells or the lysate and the normal cells or the lysate.

24. A method of treating or preventing cancer in a subject comprising increasing Chk2 activity in cancer cells of the subject in the absence of de novo DNA damage.

25. The method of claim 24 comprising selectively increasing Chk2 activity in cancer cells of the subject.

26. The method of claim 24 comprising administering to the subject a therapeutically effective amount of a Chk2 activator that is not β-lapachone.

27. The method of claim 26 comprising administering to the subject a therapeutically effective amount of a selective activator of Chk2.

28. The method of claim 26 wherein the compound is an analog, derivative, or metabolite of β-lapachone.

29. The method of claim 24, wherein the subject is a vertebrate, mammal, or human.

30. A method of treating or preventing cancer in a subject comprising administering to the subject a therapeutically effective amount of a Chk2 activator that is identified by the method of claim 1.
